SEQUENCE LISTING (65 sequences)

SEQ ID NO: 1 - DNA, 16501 bp, Homo sapiens
agatgacatctattgaaaatatccctgtctcagccaggtgcagtggctcatgcctctaatcccagcacttttggaggctgagtgagtggatcatttgaggtcaagagttcgagaccagcctggccaacatgatgaaaccctgtctctactaaaaatacaaaaattagctgaacttggtgacacatgcttgcaatcccagctactcaggaggctgaggcaggagaatcacttgactccaggagacagaggttgcagtaagccaagatcatgccacagcactccagcctgagcaacagagcaagactccatctcaaaaaaaaaaaaaatccctgtctcaaactcctgctttccaggtagatggagcgggataatgtgctttaattggtgaagggtttctgatccccatttatcatgcagaaggcaggagcaagccagagacaggtcctggcgaaaggaagctaaaatggtgcctttgatacaggggaaagggatacgaaaggggcttaggtgagacctccttacagctagtggcacctggtcagcagggccgtagagttgaggccatccccttagcagcagtgggactttgttacacacaccctctgagtcttggtgagaaactagtcccagggccccttgttgtctctgccactcccattcatgacatcatggaaacttctgtgcggctgggctcctgaacacagcagcctgccctcggctggatgaggcagccaaaaccctaaaagggttgtgatagggactggagtcaacaagtagctgtgggaacgtggtggcgcagacccttcacccagggagccctgtgggctagaaacaccagacagccaggatgtggcgagagactgcccaacacactgtctgtgtgaggcagaggaaggaaggagatgaaggaagaaacaggtaggtaggagggcgagatcccaagcaagtcaggacaactcccacgccttgcccaggagccttaagaagcccaggcacctgctgagtgaaagaggatatatttattggctgagcaagaagggaaggtacagttggtaatggctgcttctagaagccaccagtctcaggttcacttgttccgagcccagtttcctccaaggtggctgtactgagcatcatctcgatctcggaggggctaagagaggagaagagaaaacggtcaggaggcagggttagaactcttcaaggaagggccccagcaggccctgacgatgagagctcctgcctgacctctccagtcacacccagcaatgaactccccagtaggacccttcccacgtgcaaacagcattcagtgtgagcctctctatccccacttaccctcccttcattcctgcctccttcccctcaacgctcacctgatagacctggtcattcctcaacagagcttgtgtgtcggcagctagaagaaccagagagagacatcaatggcctagcagatgggactgtgagatccaccctcccacaccctcagaagtctgcatgagtgatatgaacacacaagttctttcaacagtcaaagttctaggagtctttaggagattgcaagagtaactcccagctgagactaaacctaccaccagccccattccttagctggtgcataagctcactggtacacacacacacacacacacacacacacacacaaacacactctcatgctctgctcttccactaacccccagacagccttccagtctcatgtccagcaaagcagaagactcccaaagcaaggagcagagtggcaatgacatcagtgacaatgatgccagccacggtggctggatccagctccacacagctctggcacactgtgggggaagggaggagagaggagaggttgagagcctttaagatcagggaaccatcctctgcctcctagggcaacccttagatctcttatgccaaaacccaaacttcaataagaccctgggagaaaggcctggtgatgggcttgccacaccttcctccaccctgcatcctaaggaatccctgggaagagccaagaagtaatctcagcttttgggaccttgaacaaggtggtgggccaaacctctcacccaaccaaggctgcagtaacatgacccctactgctctgtcttgctgagttaagctctctcttccactttgaaggtccccaaatctggcttgtaccattacaatagtgacctcactttgtttagagaacagatgggttcaccatatccctctctagccagaaagttctcacatccagaagccctatccattccaacccaaagggttcaggaagcacgtacttcgataatgaacttgcacggtagattctttgtccttgtatatatctgtcccattacacctatatattcctcgtgggtccaggatgcgttttcccaggtccagtcttgtaatgtctgagagcagtgttcccaccgttccctctacccatgtgatgctggtattgcaattcacaaacactctgtcctcaagttcctctataggtatcttgaaggggctcactaaaggggaaaaaatatcacagttggagacagctctttgatctgcaccaagccctttgttctgcggaagctcatacttaacagagaccattttcctggtccaggacagtttatggcttccatcaagagagacagaagtcacaagaaaaagccttcagaaagttccccaccaactgcaggggtcaagggggacatgaggatgccattcaagcagaggacaggtcttggggccttggtgcaaaagaggacccctcagagcaggattgacccaagcaccttcctggaaatgaatccagaccactgatgaggagtagggggagcacggaccactgaagcacctggaagatgtggaaagacagaagaacattcctcgattggaaatgtctgcattttttcttcaaggaaacatctaattccacttcccagccatctacaacactcccacttcagcttcttatcctcatccttcctcattgcccctgctccattgacaaccaagaaagcggggctctcaacactgaagcctttcccagggccagggatggctgtgggtggagaccagctggtttaccagcccctgaattatcagccaagtggtccagaacgggaccagggcaaatcccatgtacagttttccacccttggttagaaggaggagaacaggaaaaaaattttattgaatccatccctagagctcctcacaagtcaagtcttgtgggagacttttagggctggaggtgagtgcagcaacattccagatgcagtgagttcctctgacagcctgagcacatctccacaggccacagaggcactacagtctatgcctccaaacacagggaaaagtggaggctacattcattcatcctgggcttcacactaagtcccaaatttggatacaagagcatcttctagaaaaccctgaaacagctgttgctcacacttctgaagcaggttggaagtatatgcatgtatcctcagggagacacatgcacatcaaatgcttcacgtcctacagtcgcgtcctcttcagggatctgtctccagtggaaatcctgagtgccctagtgcagccaactattaggtgaccattggacccagtttgcttagtgttgaaggggttcctcggacatgggactttccattttaaaactgaaattggcaaactgagatgagttaaaatcctaccatgtaacaacccctcaaatcttccctccgtcctgctcaacctaaagttaacttctcttaaagcattcacataagtgctaggacatgcctccagggatgacataatcatggccaaacaaacaagagtcctgattccagaggccatcaggcctaaaaggagtagtgcaggaagctgtgctcccatggccagtcccagattcaggtacatacgtactgagctattttctgcagatctctggcctaaggccttctgagagacattctaggcccacatgcacccatggctggagtcagtcaaagccaagagcctgtttcccagactctatgctacatcctgcccctgccctcctgacacccctggggtgcctggtgaactgaagctagcaccgagaagcacttttttttttttttgagataaggtctcacaggttgcctaggttggagggcagtggcatgatcacagctcactgaagccttgaaatcccgggctcaagtgaccctcctgcctcagcctctcaaatagctgggactacagttgtgcaccaccatgcctggctaattcttttgttttttgtaaagatagagtctcatcatgttgctcagactggtctcaaactcctggcctcaaaggatcctcccacttcggcctcccaaagctctgggattactggtgtgagccaccgtgcctggcaagaaacactttcaagtgggcctcactcccatcagtaatgtccctctcaggtcccttcccccacccacctggagtagccttaccttgcgagagaagggtagccagtaccaggccagagagaaacgtgctatgttccatctcccagcggaactcatccagtagataaagccaggtcaccgaactatcagcctgggtgagagctgccctcccctagctgactcacaggtaccggaaagaggagagtgggggaggaactagaagatgtctgcttctctgctttctgccgttgatggtgggaaattttcagagtgggggtgggctaggggtagggtaggaaggaagcggtgtaaattgctctatctctgagcagggagctggcagagaatatggaaaaggtggcctgaactttagcgtccctattgacaatgcaaccacatttacacacaacctaaacactgccacatctcgaagccccttgagagaagccgtcggccccatagcgcaagccgtagcagctagatttctcatggaggctgatctttctcaggacccttcactaggcagccagggacaccagatctagcagcttcttgtcagtgggaggttgggctttagagaccccagccagagatttgaatcctgggtccaatactgcctacctgtggggcctgggccagccataaaatttttcagagtcttattccattagtaccattattaggattcaaacaagatatttgcatggtgcctcacgcatcatatgtgctcattaaggggtagttattaataataatataattgactgacaggcaatattgagcctcccggtgagacaaatggacctttttcccctgtggcctacgaggatctgaaactcttcacgctgctgcagttagactgtcacttacctggggacagagtcatgcctgtcttgctcactgctgtatcttgtgcctggcacataacgggagctctgcacatttttgttggctcactgactgactggctgagggagataggggcctgagatcctggacattcagtccgggctctggcccctgaaaatgtgctggcctgtcctcggaattgttccacctattgccttccaggcgcctctttcatgatctcaaaagaatagtgaaaccaggtgccgtgtctcacgcctgtaatcccaacactttgggaggctgaggcaggtggatcacaaggtcaggagttcgagaccagcctgaccaacaaggtgaaaccccgtctctactaaaaatacaaaaattagccgggcttgctggcacgcacctgtaatctcagctactcaggaggctgaggcaggagaatcgcttgaaccccggaggtggaggttgcagtgagctgagatagcgccactgcactccagcctgggcaacaaagtgagactctgtctcaaaaaagaaaaagaaaaaaaaagtgaaaaaaattcctgaatgaaggcctggactgaggtggctttccatttggaggtccagccccaagcatctgagagtccctcctaaattcattacctacagcaacaacaactagcaacaagtaacaactggctacgatcctaacaactaatgacagggacatttatctccctcatgaagaaacggtcccaggaccatctcccacccagcatccattgcggttccctgtgcaagatgagtctctgagtgggaatccagcactctctccctcttcttccccaccaccttcaccctccttaacggaaaaacaaaaggcatctgcacctgcagccctgctgaggcccctgctgctcacacttgcagcagagggtggaggctctgggttcttgccttctctcaaaggccccagccccaacagtgatgggtggagccagtctagctgctgcacaggctggctggctggctggctgctaagggctgctccacgcttttgccggaggacagagactgacatggaacaggggaagggcctggctgtcctcatcctggctatcattcttcttcaaggtaagggcctactaggggtctggaagcctggggaagggctcaagggaagagcccatcactagtgagacaggaatattggtatccctaaccttcagcctacctctgctgtcaccttagagttcaaagaagggcaaaatggaggctcttaactgttctctgctagagagaaacagtgtcccatggaggagaaggaatccttgtctctgaaaaatgcaaacagagtacttaaatggctgaagagaggaccctgttaccgccatcttagatttgaatgcagcccaaaagggcataggccaagaaactaaaaggaaaaagtatatgttccctacttcagagctgggggctagcagtcgacctaggaaatgtccattcactcagttgggcagttggccaattggtcaacaaagatgggtgagaccctaatatgtgatatgccatgaggaaataaaaatgaataaaacacttatgctctacagaactcatagtctcattggagagattagttttcatggtgccagactgagtgtacagtatagtatgggtgcctgcatgaatactccattggactatccaataggaagtggatgaactgggttgatgcctggagtctggacatcctgggaaagttaaaaccatggaatttgctgaagtcacacaagtactatgtgaggaagagggagctgaagatggaacagtgagggcaccaattttaaggggcagttagagaaaaaaaaacattccaggaaagagataaaaggagaatcaaagagctgaaataatcaaatgtatgcactaccttggaaaccaagagagtagagagcttctagaagaagagaattaccaagagtataaaatgttaaagagagcccactaatataagaatatatttaaaagtttaagtctgcgggatgtggcagaatatgcatcatgggtgatttagcacagtttcagcagggtattgtgagtggaagccaagtgaaaattggtagaaggtgaaggtcaggtgatgaggtcagcaagtgtagactattctttaaagacatttggatgaggagggagaaaagactgggtggtaggtagaggaggaagtagagtctaagaataaatgtttttgagggtgagagaaacttgagcacatttctaggctaaaggagaagatgatgagtagagcaaggtcctggagttgatggtggaaggacagagtcaagagtggggtcaagagcacagagaggaaggcaacgttaccctctaagaccaggagaaaggaagtgcaaacggcactggccacaaataggtttataaagggaaaggaagctaaaggagaaaggacaataacaacttccgtgttctaaaaaatccaggtgggaggcaatgccacctgctgaaactgagctgggtgatgaccggttgggagacttgaagaggacagtgagagtttagaatagaattatttctgagagaaatgcaaggaaaagtcaggtaaagacaacacaggttgaggagcaactctcaagcctgggtgaggtggctgcatatcatggaaagagcatttgagttcagcagtagaaaacctgagctctggatccagcttggacaccaatgagctatgtgtacttgaacaactcctccactgtctgggagttctcatgccagccaatccagaaatgtggcagggacgggcatggtggctcatgcctgtaatcccagcactttgggaggtcgaggcaggtggatcacctgaggtcaggagttcgagaccatcctggtcaacatggtgaaactctatctctactaaaaatattttaaaaattagtggggtgtggtggtgggtgcctgtaatcccagctactcaggaggctgaggcaggagaattgcttgaacccaggagatggaggttgcagtgagccaacacggtgccactgcactccagcctgggcgacagagtgagactccgtctcaaaaaacaaaacaaaaaaaaaagaaatgtggctatatgaactcttaagtcctaaaaggaaacctcactcagatatgcaacattaaaagatgtcaatcagccaggcacagtggctcatggctataatcccaacactttgggaggctagggagggaggatcatttgagcccaggagttcaagaccaacctgggcaaaatagggataccctgtgtctacaaaaactacgaaaattagccaggcatgttggcatgcacttatgccagctacttgggaggctgaagtgggaggattgcttgagcccaggagcttgaggctgcagtgagccatgatcacaccactgcacttcagcctgagtgacagagcaagaaaaaaaaaaaaagatgtctcaaaaaaaaaaaaaaaaactatgtcaatgatcagtgcaatctccttccctgaatccaatttagaatgaggggtctctgtcttctctcggtcttctgaccaggtggtcaggagaaaaactagttagaaacctctttaactgtttgtcttgccttttctcccatttgctcatttattcatttgcacattcattcaacaaataaagttcctaccatgttccagacactgcactggatactgttcaaaaataatcccgccctgcatgaggttctaagacagttcacttcttgtatttccattatactttgactcatgaccgcctctccctatcacacacactccacctctagccacactacctctcttgtccttttccaaagctgcatcttattcagctattctttctactcagaatgactttctctatctggtatattcttgcttactcttcaaggcccagacaaatgtcgtttcctctattaagctttccctgatcctcacccttggacaaaattaatagcttctacccttttttcatatgcagtcatataagcactaaaagataatgtatacattctattaaattaattgcatatatgttttaattttttcacttcattcataaactttggaagcagcagttgattcttcatatcagcactcttttctttcataatatctaattattttaggtcataatagtcctcgttaaatgtttggattaaatctaattgaattgaattcaagtgccagatctctgtaatatgtctaccatgcaactctactaccctaaggttttgtttgtctgtttgtttgtttttatttttttgagacagctttaacacctaggctggagtgcagtggtgcaatcttggctcactgcaacctccacctcccaggctcaaccattcctcccacctcctgagtagctgggactacaggtgcataccaccatgcccggctaatttttttgtattgtctgtagagacagggtttcgccatgttgtccaggctggtgtctaactcctgggctcaggcaatctgcccacctcagcatcccaaagtgctgggattacaggtgtgagccactgctgccagtcaactctgagttatgagacagatttgaggactaattgaaaagctaacttccctgggacatccagggtttttataataaaaggatcaccaaggctgaatattttataaggaactcagcctaaaggttttggatagtgcacacccactccttgccagggcattagctgctcaagaagcagagtgttctggactggataggcaatatccttacttacatatactacaatacaattctgatgctaaccccccaaattagtgtcagaccctacaggttaagagcatggttcccaaccagattgccctcactttaaatgccagctacaagttcagggtaccccagaccactcacatttctgacaaactgcctgcaatcttaaggattcccatgaccctcctcattgataatttgctagaataactcacagaatttaagaaagtgctgtacttcctatttcagttttattaaaaaataaaataaaatcagaaccagctaaatgaaaagggcaaggtctaggagggtcccgaacacagagcctccgtgccctctccccgtggaattagaacacatcgcccccagcactctcccacactctgccagcacattgatgggttcaccaaccaggaagctccaccaagtttttattgaggtatctttacataggcatgattgattgaatcattggccgcttgactgatctcaatctctaggatcccttcctggggctgataccactagtttcaaagctgcaatcctcttaccatatggttggtcttgaccagcaccatcctgagtcattgccatgcataaactcaggtatggtctaaggatccaccacagataacaaagccactcctgtcactcatgaaattccaacggttagaaacaccctcccaggataccaggacaaagatgagacaaattgtttattatcaccttgaattcaattgagtgatttagtctacaatccggaaaactaagtatagatactaccattttcatggatttggatctttcttcatcttggcctcaaataaccatggaaatacttcagggcatctgaacaactccatgcccagctaatactctatctctcttctgtctttacaggtactttggcccagtcaatcaaaggtaggagaaatggcttctttctatactcagactcagaatattgacggaaatttggcttcctacaacagtagtcctacaggagcgaacagtttagaatgaatgaaatgacggggatagagaggtgatgtctctattgtcaaccaaatcagtgacctgacataacctgttccgggcagcttgcctgtagctaagcatttaactggtctcttacaggagaagcaggaccctagtagctagggacacatctcaaactgtgacccatgaaccagtaggtattggcgtcacctgggatcctgacgttagtctctgtcaatccttctctttagttcatctattctacccaaagtgatctcatcatctggtatgctgttagcagtttcttacctgtatagtatcttccaaataacatgccccaaaatcccaaagttttacccctactaattacagcaatgtctcttttattcttcaccccctgacgcagatattggcgtcacccgagagcatgttagtaatgcagaatctcccctccccagaactactaaatagcacctgaaattttaacaagatccccatgtgattcatgtgcacatcaaagtttgagaaacactactctaatgatctcctggtatgcagaagcagggagaatttcagaggcaagatccttaatagaaccacggcttttctcatttcaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaagtatgtaatcccctttggtctgtttgttgtgaaattaatcagtatttgctgttctggtgagctttttatctggggtgaaagtggaaatagatcctcaacagtaatattatcgcctgttctcttaatttcagcttgcctcttttaaaatactgtaagatacttccctcaccctattgaaaaactacagccagtcctgtaaaattttgtttacctttgggtgggctccatggattcaagcaattcagcactgagttgaatgaaggggttggggagacagaggtgatcataaggtgagcagcagtgagaactggagcgcagtggagcatgaagagtatctagtcttccttctgttccagatacttcctggtagttagactacatgggcttccccaggaatcctgggggacttaagagcatcaagatgcattgagttttggcgcaaggatcttcccttgccctgccacccacagaggaaaagcctgctgccctccacagcggcattattgcagacaggcaggagaaaacgaaccaggaaaaacaactttcgcaacctgaaggtttgtctctccttttccctacagtgtgtcagaactgcattgaactaaatgcagccaccatatctggctttctctttgctgaaatcgtcagcattttcgtccttgctgttggggtctacttcattgctggacaggatggagttcgccagtcgagaggtaaaagaatgctcttagatgagagatgggaccacctgagaccctcagctttcctcctaccattatgtacccaatggaagagactgagttggtgcttcttgctagtgtgcatagttgggtgaggctgtatttctctgagacaggagaaaggatacttggtgttatcacagcatgttcacctgtctcaagatacagctccctctgtgaaaaaaaaaaaaaacaaaaacaggcgcagtggctcacgcctgtaatcacaacactttgggaggccgaggcgggcagatcacgaggtcaggagatcgagaccatcctggctagcacggtgaaaccccgtctttactaaaaatacaaaaaattagccgggcgtggtggcgggtgcctgtagtcccagctactctggaggctgaggcaggagaatggcatgaacccgggaggcggagcttgcgtgagccgagatcgtgccactgcactccagcctgggcgacagagcaagactccgtctcaaaaaaaaaaaaaaaaaaaactattttaagtaaacccttacatacagaaaagtgaatcacaagcatacagctcaaaaaaaaaattcacagagcaaatacacccatgcagccagcacataatttaagaaagacaatattaccaatgccattgaagcccctcttgtggtcacttccagccactacccatcctcaagagtgagcattgtcctgacttctaacaacaaggattaactttgcttgtttttgtcctttatgtaaatggactcatatactaagtattcttgtgagtagcttctttcactcaacaacatgtttatgtgctttatccatactcttgtgtatatataaaactcttattgttatttagtattccattttgtgaatataacccaatttattatgcattctaccttgtgtggattctgtttcttttttgtgcagcttcagacaagcagactctgttgcccaatgaccagctctaccaggtaaggggatgaagaataaaagagacattgctgtaattagtgggggtaaatctttgggattgaggggcatgttatttggaagatcctatacaggtaagaaactgctaacagcataccagatgatgagatcagtttgggtagaataaatgaactaaagagaaggattgacagagactgaggtcaggatctagtgagcacaagttgaagaacacactgagagggacacacgaagaaactctcgacaggctgggcactcactatagacaggccatggctcatgcctgtaatcccagcactttgggaagccaaaacaggtgaatcacctgaggtcaggaattcgagacaagcctggccaacatgatgaaactctgtctctactaaaaatacaaaaaaaattaaccaggcgtggaggcgcgcgcctgtagtcccagctactcaggaggctgaggcaggaggaccacttgaacccaggaggtcgaggttgcagtgagctgcgattgtgccactgcactccagcctgggcaacagagaaagactccgtctcaaaaaaaaaaaagagagagagagaaaaagaaaaaagacagagcctccatctccttgtcctctttccatcctcaggaccatgaagtacccactccaaattctcacatataaaaaacattcaataaacatgcatcaaattaattaatagaggatggaaaaaatgacttatgactgtgctgtcctttccagcccctcaaggatcgagaagatgaccagtacagccaccttcaaggaaaccagttgaggaggaattgaactcaggactcagagtaggtgggttcttcaatgccaattctaataaaggacccttgcatcaactgccctcgcaattgcttctaagtctagctcccttccctaagcggctataagcatcagactctggggatcagggattgggacgtggtttggggtactcttttctaaaaattctggggccatactgattgtcttggcctaggtaaatatgaattttatgtatctgtaaatcctgtcagagcagggcctcaagccatagagatgctgaatattaatcttaacctacatttgaatttctcattatctacactattaacattttgggctaattaattatttgtgatgaggggctagcctgtgcattgtaggagttatggaagcatccctggcctctctccaccagatgctggtagattgtccagtgtgacaatcaaaaatgtgtccagacattaccaaatgtgtccaaacatcacctccagggcaaaatcacccttagttaagaaccactaacccatattaaccttccaatcaataaatcaatcagtcagaagttatgatttaattaatctatctgaagtttctatcaggaagacagggttgaaagcattatttgttttttttgaacaaattgcaatttttcttttttcagtccaggtgttctcctcctattcagttcccagaatcaaagcaatgcattttggaaagctcctagcagagagactttcagccctaaatctagactcaaggttcccagagatgacaaatggagaagaaaggccatcagagcaaatttgggggtttctcaaataaaataaaaataaaaacaaatactgtgtttcagaagcgccacctattggggaaaattgtaaaagaaaaatgaaaagatcaaataaccccctggatttgaatataattttttgtgttgtaatttttatttcgtttttgtataggttataattcacatggctcaaatattcagtgaaagctctccctccaccgccatcccctgctacccagtgaccctgttgccctcttcagagacaaattagtttctcttttttttttttttttttttttttttgagacagtctggctctgtcacccaggctgaaatgcagtggcaccatctcggctcactgcaacctctgcctcctgggttcaagcgattctcctgcctcagcctcccgggcagctgggattacaggcacacactaccacacctggctaatttttgtatttttagtagagacagggttttgctctgttggccaagctggtctcgaactcctgacctcaagtgatccgcccgcctcagcctcccaaagtgctgggattacaggtgtgagccaccatgcctggtcttaaaaccagtttcttatatatctctctggaggtattctaggcatatatgagcacattctcaagtacatattatcctcccttcccctatcttttagacaaatgatatcaaactatacatcttgtgagattattgcataccattatatgaagataccattatatcctttttaatgcaaccatattgtacaaatagactatgatttatttaacctgttatctatcagtggatatttaagttggtagttggttccaatcttttgctcttacaacaattctgcaatgactaacattgtataaatatcatttttaaaaataattgcattgaagcataatgtacatgccataaaatccacccatcttaagtgatttcacctgttctcagaaatttttagtaaatttaactaattgtacagccattaccataatccagctttaggacattttcttttttttcttttcttttcttttttttcttttttttttttttttgaagtggaatcttgctctgtggcccaggctggagtgcagtggcgcgatctcagctcactgcaacctccacctcctgggttcaagcgattctcttgccttggcctcccgagtagctgagactacaggcacatgccaccacgcccagctcattttttgtgtatttagtatttgtgtatctagtatttgtgtacttagtagagacagggtttcaccatgttggccaggctggtctccaattcctgacctcaggcgatc

SEQ ID NO: 2 - DNA, 50 bp, Homo sapiens
agctgctgcacaggctggctggctggctggctgctaagggctgctccacg

SEQ ID NO: 3 - DNA, 50 bp, Homo sapiens
cggaaaaacaaaaggcatctgcacctgcagccctgctgaggcccctgctg

SEQ ID NO: 4 - DNA, 50 bp, Homo sapiens
acccaggctgatagttcggtgacctggctttatctactggatgagttccg

SEQ ID NO: 5 - DNA, 50 bp, Homo sapiens
tggaacatagcacgtttctctctggcctggtactggctacccttctctcg

SEQ ID NO: 6 - DNA, 435 bp, Homo sapiens
ttattccacctattaccttccaaacgcctctttcataatctcaaaaaaataataaaaccaaataccgtatctcacgcctataatcccaacactttaaaaaactaaaacaaataaatcacaaaatcaaaaattcgaaaccaacctaaccaacaaaataaaaccccgtctctactaaaaatacaaaaattaaccgaacttactaacacgcacctataatctcaactactcaaaaaactaaaacaaaaaaatcgcttaaaccccgaaaataaaaattacaataaactaaaataacgccactacactccaacctaaacaacaaaataaaactctatctcaaaaaaaaaaaaaaaaaaaaaaataaaaaaaattcctaaataaaaacctaaactaaaataactttccatttaaaaatccaaccccaaacatctaaaaatc

SEQ ID NO: 7 - DNA, 438 bp, Homo sapiens
ataaacctcactcccatcaataatatccctctcaaatcccttcccccacccacctaaaataaccttaccttacgaaaaaaaaataaccaataccaaaccaaaaaaaaacgtactatattccatctcccaacgaaactcatccaataaataaaaccaaatcaccgaactatcaacctaaataaaaactaccctcccctaactaactcacaaataccgaaaaaaaaaaaataaaaaaaaaactaaaaaatatctacttctctactttctaccgttaataataaaaaattttcaaaataaaaataaactaaaaataaaataaaaaaaaaacgatataaattactctatctctaaacaaaaaactaacaaaaaatataaaaaaaataacctaaactttaacgtccctattaacaatacaaccacatttacacacaacctaaa

SEQ ID NO: 8 - DNA, 472 bp, Homo sapiens
aatccctcctaaattcattacctacaacaacaacaactaacaacaaataacaactaactacgatcctaacaactaataacaaaaacatttatctccctcataaaaaaacgatcccaaaaccatctcccacccaacatccattacgattccctatacaaaataaatctctaaataaaaatccaacactctctccctcttcttccccaccaccttcaccctccttaacgaaaaaacaaaaaacatctacacctacaaccctactaaaacccctactactcacacttacaacaaaaaataaaaactctaaattcttaccttctctcaaaaaccccaaccccaacaataataaataaaaccaatctaactactacacaaactaactaactaactaactactaaaaactactccacgcttttaccgaaaaacaaaaactaacataaaacaaaaaaaaaacctaactatcctcatcct

SEQ ID NO: 9 - DNA, 50 bp, Homo sapiens
agtcatctgttttgctttttttccagaagtagtaagtctgctggcctccg

SEQ ID NO: 10 - DNA, 50 bp, Homo sapiens
cgcccagcctcgctgttcttatcttggcagcagattccgaatgtcggctg

SEQ ID NO: 11 - DNA, 50 bp, Homo sapiens
cgctcatcaggacttcagcactgcccgtccatggggacgtctgcactcac

SEQ ID NO: 12 - DNA, 50 bp, Homo sapiens
cgccttgagctgtacctggcacatctttgttgctcagcaaagagtggttg

SEQ ID NO: 13 - DNA, 50 bp, Homo sapiens
tcttcccatctgcgtcttggttaaggacttttatcgtatcctgtttatcg

SEQ ID NO: 14 - DNA, 50 bp, Homo sapiens
aagatctgctggaggccaggcacagacaggtgctttaactcaagttatcg

SEQ ID NO: 15 - DNA, 50 bp, Homo sapiens
agtaactgcagcaatgagtgcccgggctgtgcttggagtaccagtgctcg

SEQ ID NO: 16 - DNA, 50 bp, Homo sapiens
cgccccaagtactaagtggcacagcttggtgagcaaggacatttttattg

SEQ ID NO: 17 - DNA, 50 bp, Homo sapiens
cggcaagatgaaacaagcttattaggctctgtcttttaagggcataccag

SEQ ID NO: 18 - DNA, 50 bp, Homo sapiens
cgcaacacagggctgcctcccccggtatatgggccccactccacagaggt

SEQ ID NO: 19 - DNA, 50 bp, Homo sapiens
cggcagagcacacagctgcagaagtaaaaaggattgaaacatttggatcc

SEQ ID NO: 20 - DNA, 50 bp, Homo sapiens; identical to SEQ ID NO: 4

SEQ ID NO: 21 - DNA, 50 bp, Homo sapiens; identical to SEQ ID NO: 5

SEQ ID NO: 22 - DNA, 50 bp, Homo sapiens; identical to SEQ ID NO: 2

SEQ ID NO: 23 - DNA, 50 bp, Homo sapiens; identical to SEQ ID NO: 3

SEQ ID NO: 24 - DNA, 50 bp, Homo sapiens; identical to SEQ ID NO: 9

SEQ ID NO: 25 - DNA, 50 bp, Homo sapiens
cgctgcagtcagactccaaagtcaggaacgtgagggctaccatctctcaa

SEQ ID NO: 26 - DNA, 50 bp, Homo sapiens
cgaaggcccccagctgtgggagcccacgctatttattggtgatcaaagaa

SEQ ID NO: 27 - DNA, 50 bp, Homo sapiens
cgcaggcacttacgatcctgccgtcgccagagcccagatccaccgttttt

SEQ ID NO: 28 - DNA, 50 bp, Homo sapiens
gggctcccgggctgggcaggtaaggagcgctggtattgggggcgcaggcg

SEQ ID NO: 29 - DNA, 50 bp, Homo sapiens
ttccgcccacaattttctgataacagccttcaaggcctcagttgctgtcg

SEQ ID NO: 30 - DNA, 50 bp, Homo sapiens
cgggaccctgttgctgactgctcaagaggaggcaagctggatctctctta

SEQ ID NO: 31 - DNA, 50 bp, Homo sapiens
taaatgatgtcttggggctgtggcccgagctgcctcaggtagatcccacg

SEQ ID NO: 32 - DNA, 50 bp, Homo sapiens
ggggactgtgggtcctcttaggggctgtgacgctgctatttctcatctcg

SEQ ID NO: 33 - DNA, 50 bp, Homo sapiens
cgcactcagcaaggcctcctgccctgagagaggctccgcccactaccccc

SEQ ID NO: 34 - DNA, 50 bp, Homo sapiens
ctcatgaagcaaggtctgcctctacttctgtgatgtggttcatgtttccg

SEQ ID NO: 35 - DNA, 50 bp, Homo sapiens
gacctttagcctcaaactcccgtggtggaaacagttaggattggtggacg

SEQ ID NO: 36 - DNA, 50 bp, Homo sapiens
gagagacaatacccttccaaaatacacttcaacataaagttttcttttcg

SEQ ID NO: 37 - DNA, 50 bp, Homo sapiens
cggtatacctgccctacttcggtatagtccttggcacatggcaggcactc

SEQ ID NO: 38 - DNA, 50 bp, Homo sapiens
atggtagccccttctgcggggagcacacaacagtcttcagttcttctgcg

SEQ ID NO: 39 - DNA, 402 bp, Homo sapiens
agcctctttataatctcaaaaaaataataaaaccaaataccgtatctcacgcctataatcccaacactttaaaaaactaaaacaaataaatcacaaaatcaaaaattcgaaaccaacctaaccaacaaaataaaaccccgtctctactaaaaatacaaaaattaaccgaacttactaacacgcacctataatctcaactactcaaaaaactaaaacaaaaaaatcgcttaaaccccgaaaataaaaattacaataaactaaaataacgccactacactccaacctaaacaacaaaataaaactctatctcaaaaaaaaaaaaaaaaaaaaaaaaaaaaaaatttctaaaaaaaaacccaaaaaaaaaaaacttttcctttaaaaaaccccccccaaaaattt

SEQ ID NO: 40 - DNA, 394 bp, Homo sapiens
agcctctttataatctcaaaaaaataataaaaccaaataccgtatctcacgcctataatcccaacactttaaaaaactaaaacaaataaatcacaaaatcaaaaattcgaaaccaacctaaccaacaaaataaaaccccgtctctactaaaaatacaaaaattaaccgaacttactaacacgcacctataatctcaactactcaaaaaactaaaacaaaaaaatcgcttaaaccccgaaaataaaaattacaataaactaaaataacgccactacactccaacctaaacaacaaaataaaactctatctcaaaaaaaaaaaaaaaaaaaaaaaaaaaaaaatttccaaaaaaaaacccaaaaaaaaaaaactttccctttaaaaaacccccccc

SEQ ID NO: 41 - DNA, 435 bp, Homo sapiens; identical to SEQ ID NO: 6

SEQ ID NO: 42 - DNA, 399 bp, Homo sapiens
cgcctctttataatctcaaaaaaataataaaaccaaataccgtatctcacgcctataatcccaacactttaaaaaactaaaacaaataaatcacaaaatcaaaaattcgaaaccaacctaaccaacaaaataaaaccccgtctctactaaaaatacaaaaattaaccgaacttactaacacgcacctataatctcaactactcaaaaaactaaaacaaaaaaatcgcttaaaccccgaaaataaaaattacaataaactaaaataacgccactacactccaacctaaacaacaaaataaaactctatctcaaaaaaaaaaaaaaaaaaaaaaaaaaaaaaatttccaaaaaaaaacccaaaaaaaaaaaactttccctttaaaaaaccccccccaaaaa

SEQ ID NO: 43 - DNA, 394 bp, Homo sapiens
tacctctttataaatctcaaaaaaataataaaaccaaataccatatctcacacctataatcccaacactttaaaaaactaaaacaaataaatcacaaaatcaaaaattcaaaaccaacctaaccaacaaaataaaaccccatctctactaaaaatacaaaaattaaccaaacttactaacacacacctataatctcaactactcaaaaaactaaaacaaaaaaatcacttaaaccccaaaaataaaaattacaataaactaaaataacaccactacactccaacctaaacaacaaaataaaactctatctcaaaaaaaaaaaaaaaaaaaaaaaaaaaaaaatttccaaaaaaaaacccaaaaaaaaaaaactttccctttaaaaacccccccc

SEQ ID NO: 44 - DNA, 396 bp, Homo sapiens
gacctctttataatctcaaaaaaataataaaaccaaataccatatctcacacctataatcccaacactttaaaaaactaaaacaaataaatcacaaaatcaaaaattcaaaaccaacctaaccaacaaaataaaaccccatctctactaaaaatacaaaaattaaccaaacttactaacacacacctataatctcaactactcaaaaaactaaaacaaaaaaatcacttaaaccccaaaaataaaaattacaataaactaaaataacaccactacactccaacctaaacaacaaaataaaactctatctcaaaaaaaaaaaaaaaaaaaaaaaaaaaaaaattcccaaaaaaaaacccaaaaaaaaaaaactttccctttaaaaacccccccccaa

SEQ ID NO: 45 - DNA, 387 bp, Homo sapiens
cgcctctttatatctaaaaaaatataaaccaataccgtatctcacacctataatcccaacacttaaaaaactaaaacaaataaatcacaaaatcaaaaattcagaaaccaacctaaccaacaaaataaaaccccgtctctactaaaaatacaaaaattaaccaaacttactaacacacacctataatctcaactactcaaaaaactaaaacaaaaaaatcacttaaaccccaaaaataaaaattacaataaactaaaataacaccactacactccaacctaaacaacaaaataaaactctatctcaaaaaaaaaaaaaaaaaaaaaaaaaaaaaaatttccaaaaaaaaacccaaaaaaaaaaaactttccctttaaaaaacccccc

SEQ ID NO: 46 - DNA, 391 bp, Homo sapiens
atgattgttaatagggacgttaaagtttaggttattttttttatattttttgttagttttttgtttagagatagagtaatttatatcgttttttttttattttatttttagtttatttttattttgaaaattttttattattaacggtagaaagtagagaagtagatattttttagttttttttttattttttttttttcggtttttgggatttatttaggggggggtgttttttttttgggttgatagttcggggatttggttttatttatgggaggatttccgtggggaaaggaaaaaaaaacctttttttttgggttgggtttgggttattttttttccgaagggaaaggttttttaagggggggggggaaagggttttaaaaggaaa

SEQ ID NO: 47 - DNA, 328 bp, Homo sapiens
ctatttgatgggagaagtgaggtgttaataagtttaggttattttttttatattttttgttagttttttgtttagagatagagtaatttatattgttttttttttattttatttttagtttatttttattttgaaaattttttattattaatggtagaaagtagagaagtagatattttttagttttttttttattttttttttttgggtttttgggatttatttgggggggggtgttttttttttgggttgatatttgggggattgggtttttttaatgggaggatttttgtggggaaaggaaataaaattttttttttttgggttg

SEQ ID NO: 48 - DNA, 388 bp, Homo sapiens
ctatttgatgggagaagtgaggttgttaataagtttaggttattttttttatattttttgttagttttttgtttagagatagagtaatttatattgttttttttttattttatttttagtttatttttattttgaaaattttttattattaatggtagaaagtagagaagtagatattttttagttttttttttattttttttttttgggattttggaattaattaaggggggggaattttttttttaggttgaaatttgggggattgggttttatttttgggaggatttttgtggggaaaggaaaaaaatttttttttttttgggttgggtttgggttttttttttttggaagggaagggtttttttggggggggggggaaggggtt

SEQ ID NO: 49 - DNA, 401 bp, Homo sapiens
cattgatgggaagtagaggattgataaagtttaggttattttttttatattttttgttagttttttgtttagagatagagtaatttatattgttttttttttattttatttttagtttatttttattttgaaaattttttattattaatggtagaaagtagagaagtagatattttttagttttttttttattttttttttttgggtttttggaattaatttgggggggggaattttttttttaggtggaaatttgggggattgggttttatttatgggagaatttttgtggggaaaggaaataaatttttttttttttgggttgggtttgggttttttttttttgggagggaagggtttttttgggggggggggggaggggtttaaaggaattttttggg

SEQ ID NO: 50 - DNA, 387 bp, Homo sapiens
attggtatgtgttaagtatgggatgtgtaaagtttaggttattttttttatattttttgttagttttttgtttagagatagagtaatttatattgttttttttttattttatttttagtttatttttattttgaaaattttttattattaatggtagaaagtagagaagtagatattttttagttttttttttattttttttttttgggtttttggaattaattaaggggggggaatttttttttaaggtggaaagttgggggattgggttttatttatgggaggatttttgtggggaaaggaaaaaaatttttttttttttgggttgggtttgggttttttttttttggaagggaagggtttttttggggggggggggaaggggtt

SEQ ID NO: 51 - DNA, 420 bp, Homo sapiens
attgttaatagggacgttaaagtttaggttattttttttatattttttgttagttttttgtttagagatagagtaatttatatcgttttttttttattttatttttagtttatttttattttgaaaattttttattattaacggtagaaagtagagaagtagatattttttagttttttttttatttttttttttccggaatttgtgagttagtagggggagggtagtttttatttaggttgatagttcggtgatttggtttaatttattgaatgagtttcgttggggagatgaaatatagtacggtttttttttggtttgggtatgggtatttttttttcggaaagggtaaagggtattttaggtggggtgggggaaagggattttggagagggattatattgatggggagtgaaggtt

SEQ ID NO: 52 - DNA, 416 bp, Homo sapiens
tatccctctcaaatcccttcccccacccacctaaaataaccttaccttacgaaaaaaaaataaccaataccaaaccaaaaaaaaacgtactatattccatctcccaacgaaactcatccaataaataaaaccaaatcaccgaactatcaacctaaataaaaactaccctcccctaactaactcacaaataccgaaaaaaaaaaaataaaaaaaaacctaaaaaatttttccttctttcctttctcccgttaaaaaaaaaaaattttcaaaaaaaaaataacctaaaaaaaaaaaaaaaaaaaaccgttttaatttcctttttttttaaccaaaaaattaacaaaaaatttaaaaaaaatacccaaaattttaccgcccctttaaacaatccacccacttttccccacacctaaaaa

SEQ ID NO: 53 - DNA, 416 bp, Homo sapiens
tatccctctcaaatcccttcccccacccacctaaaataaccttaccttacgaaaaaaaaataaccaataccaaaccaaaaaaaaacgtactatattccatctcccaacgaaactcatccaataaataaaaccaaatcaccgaactatcaacctaaataaaaactaccctcccctaactaactcacaaataccgaaaaaaaaaaaataaaaaaaaaactaaaaaatttttccttctttcctttctcccgttaaaaaaaaaaaattttcaaaaaaaaaataacctaaaaataaaaaaaaaaaaaaccgttttaatttcctttttttttaaccaaaaacttaacaaaaaatttaaaaaaaaaaccctaacttttaccgcccctttaaacaatccacccccttttccccccaccctaaaa

SEQ ID NO: 54 - DNA, 418 bp, Homo sapiens
gtatccctctcaaatcccttcccccacccacctaaaataaccttaccttacgaaaaaaaaataaccaataccaaaccaaaaaaaaacgtactatattccatctcccaacgaaactcatccaataaataaaaccaaatcaccgaactatcaacctaaataaaaactaccctcccctaactaactcacaaataccgaaaaaaaaaaaataaaaaaaaaactaaaaaattttttttttttttttttttcccgttaataaaaaaaatttttaaaaataaaaataacctaaaaataaaataaaaaaaaaccgttttaatttcctttttttttaaccaaaaacttaccaaaaaatttaaaaaaaataccctaacttttaccgcccctttaaaaaaaccacccccttttccccccacccaaaaaa

SEQ ID NO: 55 - DNA, 419 bp, Homo sapiens
gccatcttacccacaacccttaaccccacccacctaaaataaccttaccttacaaaaaaaaaataaccaataccaaaccaaaaaaaaacatactatattccatctcccaacaaaactcatccaataaataaaaccaaatcaccaaactatcaacctaaataaaaactaccctcccctaactaactcacaaatcccaaaaaaaaaaaaataaaaaaaaacctaaaaattttttttttctttcttttccccctttaaaaaaaaaaatttttaaaaaaaaaaaaaacctaaaaaaaaaaaaaaaaaaaaccattttaatttcttttttttttaaccaaaaacttacaaaaaattttaaaaaaaaaccccaaactttaaccccccttttaaaaaaaccccccctttttccccccccccaaaaa

SEQ ID NO: 56 - DNA, 418 bp, Homo sapiens
gccatcttacccacaaccctaaccccacccacctaaaataaccttaccttacaaaaaaaaaataaccaataccaaaccaaaaaaaaacatactatattccatctcccaacaaaactcatccaataaataaaaccaaatcaccaaactatcaacctaaataaaaactaccctcccctaactaactcacaaatcccaaaaaaaaaaaaataaaaaaaaacctaaaaattttctctttctctcttttcccccttaaaaaaaaaaaatttttaaaaaaaaaaaaaacctaaaaaaaaaaaaaaaaaaaaccattttaatttcttttttttttaacaaaaaacttacaaaaaattttaaaaaaaaaacccaaacttttaccccccttttaaaaaaaccccccccttttccccccccccaaaaa

SEQ ID NO: 57 - DNA, 419 bp, Homo sapiens
tatatcctactcaacatccctatcccccacccacctaaaataaccttaccttacaaaaaaaaaataaccaataccaaaccaaaaaaaaacatactatattccatctcccaacaaaactcatccaataaataaaaccaaatcaccaaactatcaacctaaataaaaactaccctcccctaactaactcacaaatcccaaaaaaaaaaaaaaaaaaaaaaacctaaaaattttttctttttttctttttccccttaaaaaaaaaaaatttttaaaaaaaaaaaaaacctaaaaaaaaaaaaaaaaaaaacctttttaatttctcctttctttaaccaaaaacttaccaaaaattttaaaaaaaatccccaaattttaaccccccttttaaaaaaaccacccctttttccccccccccaaaa

SEQ ID NO: 58 - DNA, 416 bp, Homo sapiens
catccctctctaatcccttcccccacccacctaaaataaccttaccttacaaaaaaaaaataaccaataccaaaccaaaaaaaaacatactatattccatctcccaacaaaactcatccaataaataaaaccaaatcaccaaactatcaacctaaataaaaactaccctcccctaactaactcacaaatcccaaaaaaaaaaaaataaaaaaaaacctaaaaaatttttctttttttctttttccccttaaaaaaaaaaaatttttaaaaaaaaaattaacttaaaataaaaaaaaaaaaaaaccattttaattttttttttttttaacaaaaaacttaccaaaaattttaaaaaaaaaccccaaacttttaccccccttttaaaaaacccccccctttttccccccccccaaaaa

SEQ ID NO: 59 - DNA, 438 bp, Homo sapiens
ctataacaacaacaactaacaacaaataacaactaactacaatcctaacaactaataacaaaaacatttatctccctcataaaaaaacaatcccaaaaccatctcccacccaacatccattacaattccctatacaaaataaatctctaaataaaaatccaacactctctccctcttcttccccaccaccttcaccctccttaacaaaaaaacaaaaaacatctacacctacaaccctactaaaacccctactactcacacttacaacaaaaaataaaaactctaaattcttaccttctctcaaaaaccccaaccccaacaataataaataaaaccaatctaactactacacaaactaactaactaactaactacaaaaaactactccttacttttaccaaaaaacacaaactaacataaaacaaaaaaaaaacctat

SEQ ID NO: 60 - DNA, 442 bp, Homo sapiens
ctataacaacaacaactaacaacaaataacaactaactacaatcctaacaactaataacaaaaacatttatctccctcataaaaaaacaatcccaaaaccatctcccacccaacatccattacaattccctatacaaaataaatctctaaataaaaatccaacactctctccctcttcttccccaccaccttcaccctccttaacaaaaaaacaaaaaacatctacacctacaaccctactaaaacccctactactcacacttacaacaaaaaataaaaactctaaattcttaccttctctcaaaaaccccaaccccaacaataataaataaaaccaatctaactactacacaaactaactaactaactaactacaaaaaactactccttacttttaccaaaaaacacaaactaacataaaacaaaaaaaaaacctatcttt

SEQ ID NO: 61 - DNA, 445 bp, Homo sapiens
ctataacaacaacaactaacaacaaataacaactaactacaatcctaacaactaataacaaaaacatttatctccctcataaaaaaacaatcccaaaaccatctcccacccaacatccattacaattccctatacaaaataaatctctaaataaaaatccaacactctctccctcttcttccccaccaccttcaccctccttaacaaaaaaacaaaaaacatctacacctacaaccctactaaaacccctactactcacacttacaacaaaaaataaaaactctaaattcttaccttctctcaaaaaccccaaccccaacaataataaataaaaccaatctaactactacacaaactaactaactaactaactacaaaaaactactccttacttttaccaaaaaacacaaactaacataaaacaaaaaaaaaacctatctttcct

SEQ ID NO: 62 - DNA, 437 bp, Homo sapiens
gtataacaacaacaactaacaacaaataacaactaactacaatcctaacaactaataacaaaaacatttatctccctcataaaaaaacaatcccaaaaccatctcccacccaacatccattacaattccctatacaaaataaatctctaaataaaaatccaacactctctccctcttcttccccaccaccttcaccctccttaacaaaaaaacaaaaaacatctacacctacaaccctactaaaacccctactactcacacttacaacaaaaaataaaaactctaaattcttaccttctctcaaaaaccccaaccccaacaataataaataaaaccaatctaactactacacaaactaactaactaactaactacaaaaaactactccttacttttaccaaaaaacacaaactaacataaaacaaaaaaaaaaccta

SEQ ID NO: 63 - DNA, 450 bp, Homo sapiens
tataacaacaacaactaacaacaaataacaactaactacgatcctaacaactaataacaaaaacatttatctccctcataaaaaaacgatcccaaaaccatctcccacccaacatccattacgattccctatacaaaataaatctctaaataaaaatccaacactctctccctcttcttccccaccaccttcaccctccttaacgaaaaaacaaaaaacatctacacctacaaccctactaaaacccctactactcacacttacaacaaaaaataaaaactctaaattcttaccttctctcaaaaaccccaaccccaacaataataaataaaaccaatctaactactacacaaactaactaactaactaactactaaaaactactgcacgcttttaccgaaaaacacaaactaacataaaacaaaaaaaaaacctatctttccccaacct

SEQ ID NO: 64 - DNA, 446 bp, Homo sapiens
tataacaacaacaactaacaacaaataacaactaactacgatcctaacaactaataacaaaaacatttatctccctcataaaaaaacgatcccaaaaccatctcccacccaacatccattacgattccctatacaaaataaatctctaaataaaaatccaacactctctccctcttcttccccaccaccttcaccctccttaacgaaaaaacaaaaaacatctacacctacaaccctactaaaacccctactactcacacttacaacaaaaaataaaaactctaaattcttaccttctctcaaaaaccccaaccccaacaataataaataaaaccaatctaactactacacaaactaactaactaactaactactaaaaactactgcacgctttgaccgaaaaacaaaaactaacataaaacaaaaaaaaaacctatctttcccct

SEQ ID NO: 65 - DNA, 448 bp, Homo sapiens
gataacaacaacaactaacaacaaataacaactaactacgatcctaacaactaataacaaaaacatttatctccctcataaaaaaacgatcccaaaaccatctcccacccaacatccattacgattccctatacaaaataaatctctaaataaaaatccaacactctctccctcttcttccccaccaccttcaccctccttaacgaaaaaacaaaaaacatctacacctacaaccctactaaaacccctactactcacacttacaacaaaaaataaaaactctaaattcttaccttctctcaaaaaccccaaccccaacaataataaataaaaccaatctaactactacacaaactaactaactaactactaacaactacttactgcttgctccgaccaacaaacactaacttaaaacaaaaaaaaaacaaacctatctttctcccaa

(19) United States
(12) Patent Application Publication
(10) Pub. No.: US 2012/0107810 A1
(43) Pub. Date: May 3, 2012

(54) Epigenetic Markers for the Identification of Blood Sub-Cells of Type 1

(76) Inventors: Sven Olek, Berlin (DE); Ivana Turbachova, Berlin (DE)

(21) Appl. No.: 13/139,808

(22) PCT Filed: Dec. 8, 2009
(86) PCT No.: PCT/EP2009/008764

§ 371 Date: Oct. 18, 2011

(30) Foreign Application Priority Data
Dec. 16, 2008 (EP) 08021838.1

(51) Int. Cl. C12Q 1/68; C07H 21/04

(52) U.S. Cl. 435/6.11; 536/23.1

(57) ABSTRACT

The present invention relates to a method, in particular an in vitro method, for identifying CD3CD4 positive T lymphocytes of a mammal, wherein said method comprises analysing the methylation status of at least one CpG position in the CD3a/b/c/d/g genes, in particular their “upstream” regulatory regions, and in particular the promoter and other conserved regions of the gene cd3, wherein a demethylation of at least one CpG in the analyzed sample to at least 90% is indicative for memory and naive CD4 or/and memory and/or native T lymphocytes. Furthermore, the present invention is directed at the use of DNA-methylation analysis of the genes CD3a/b/c/d for the detection and quality assurance and control of T lymphocytes. Furthermore, the present invention relates to a kit for performing the above methods as well as respective uses thereof. In a preferred embodiment, the present invention furthermore provides an improved method for analysing the methylation status of at least one CpG position in the gene CD3, allowing for a precise analysis even from sub-optimal quality samples, such as non-freshly obtained blood or serum samples.

The present invention relates to a method, in particular an in vitro method, for identifying CD3CD4 and/or CD3CD8 positive T lymphocytes of a mammal, wherein said method comprises analysing the methylation status of at least one CpG position in the CD3δ/γ/ε genes, in particular their “upstream” regulatory regions, and in particular the promoter and other conserved regions of the gene for CD3, wherein a demethylation of at least one CpG in the analyzed sample to at least 90% is indicative for memory and naive CD4⁺ T lymphocytes and memory and naive CD8⁺ T lymphocytes. The present invention is further related at analyzing the methylation status of at least one CpG position in the genes SLA2, CHRNA3, C16orf24, LCK, FASLG, CD7, SIT1, IL32, CXCR6, UBASH3A, GRAP2, ITGB7 and TXK which also allows the unambiguously identification of all CD3 positive T lymphocytes. For further un-ambiguous identification of all CD8 cells, also the equivalent analysis GNGT2, CRTAM, IL2RB and ZBTB32 can be employed. Among the CD3 positive T lymphocytes, these markers are capable to segregate between CD8 and CD4 positive cells. Equivalently, FLJ00060, FLJ38379, PPP6C, CD226, ZBTB7B and TNFAIP8 are capable of positively identifying CD4 expressing cells in whole blood and segregate between CD4 and CD8 positive CD3 positive cells.

Furthermore, the present invention is directed at the use of DNA-methylation analysis of the genes CD3γ/δ/ε or SLA2, CHRNA3, C16orf24, LCK, FASLG, CD7, SIT1, IL32, CXCR6, UBASH3A, GRAP2, ITGB7 and TXK or GNGT2, CRTAM, IL2RB and ZBTB32. or FLJ00060, FLJ38379, PPP6C, CD226, ZBTB7B and TNFAIP8 for the detection and quality assurance and control of T lymphocytes. Furthermore, the present invention relates to a kit for performing the above methods as well as respective uses thereof. In a preferred embodiment, the present invention furthermore provides an improved method for analysing the methylation status of at least one CpG position in the gene CD3, allowing for a precise analysis even from sub-optimal quality samples, such as non-freshly obtained blood or serum samples. It is one aim of this invention to provide a novel, more robust means to quantitatively detect and measure particular subsets of the blood within any solid organs or any body fluid of a mammal.

Employing this method, the inventors provide for novel, not previously known means of determining, quantitating and routinely measuring T lymphocytes.

### BACKGROUND OF THE INVENTION

T-lymphocytes are a major component of the mammalian immune system. Both CD4 and CD8 T-cells are responsible for proper functioning of said immune system. Whereas CD8 T-cells mediate the cytotoxic immune defence, CD4 cells—the so called helper T-cells—assist both the humoral and the cell mediated immune defence. The heterodimeric T-cell antigen receptor (TCR) is bound to a monomorphic protein complex called CD3. CD3 consists of sub-elements CD3γ, -δ, and -ε, and is expressed on all peripheral T-cells, but only on a subset of the thymocytes. The genes for CD3γ, -δ, and -68 are encoded on neighbouring loci on chromosom 11 (11q23).

Role of CD3 in signal transduction—The function of the CD3-chains is both the formation and the transport of the full CD3 complex to the cell surface, as well as the signal transduction upon stimulation through the heterodimeric T-cell antigen receptor. The amino acid sequence of the cytoplasmatic part of CD3γ, -δ, -ε each contains a motif that becomes phosphorylated upon stimulation, and thus activated. This so called ITAM (immunoreceptor tyrosine-based activated motif) serves as docking point for different tyrosine kinases. Individuals with defective CD3γ or -ε-chains suffer from a severe clinical autoimmune deficiency.

Even though almost all cells in an individual contain the exact same complement of DNA code, higher organisms must impose and maintain different patterns of gene expression in the various types of tissue. Most gene regulation is transitory, depending on the current state of the cell and changes in external stimuli. Persistent regulation, on the other hand, is a primary role of epigenetics—heritable regulatory patterns that do not alter the basic genetic coding of the DNA. DNA methylation is the archetypical form of epigenetic regulation; it serves as the stable memory for cells and performs a crucial role in maintaining the long-term identity of various cell types.

The primary target of methylation is the two-nucleotide sequence Cytosine-Guanine (a ‘CpG site’); within this context cytosine (C) can undergo a simple chemical modification to become 5-methyl-cytosine. In the human genome, the CG sequence is much rarer than expected, except in certain relatively dense clusters called ‘CpG islands’. CpG islands are frequently associated with gene promoters, and it has been estimated that more than half of the human genes have CpG islands (Antequera and Bird, Proc Natl Acad Sci USA 90: 11995-9, 1993).

Aberrant methylation of DNA is frequently associated with the transformation from healthy to cancerous cells. Among the observed effects are genome-wide hypomethylation, increased methylation of tumour suppressor genes, and hypomethylation of many oncogenes (reviewed, for example, by Jones and Laird, Nature Genetics 21:163-167, 1999; Esteller, Oncogene 21:5427-5440, 2002; and Laird, Nature Reviews/Cancer 3:253-266, 2003). Methylation profiles have been recognized to be tumour specific (i.e., changes in the methylation pattern of particular genes or even individual CpGs are diagnostic of particular tumour types), and there is now an extensive collection of diagnostic markers for bladder, breast, colon, oesophagus, stomach, liver, lung, and prostate cancers (summarized, for example, by Laird, Nature Reviews/Cancer 3:253-266, 2003).

Flanagan et al. (in Flanagan B F, Wotton D, Tuck-Wah S, Owen M J. DNase hypersensitivity and methylation of the human CD3G and D genes during T-cell development. Immunogenetics. 1990;31(1):13-20.) describe that the mouse and human CD3G and D genes are organized in opposite transcriptional orientation, their 5′ ends being separated by about 1.6 kilobases (kb) of DNA. The molecular basis of the tissue-specific regulation of expression of the human CD3G and D genes were examined using DNase I hypersensitivity and CpG methylation analysis in the mouse. The authors try to define areas 3′ to the D gene and within the intergenic region which contain regulatory elements that influence both CD3D and G expression and show that transcription from the CD3D and G genes may occur initially from a methylated promoter. Significantly, the 3′ regulatory region was shown to adopt an open chromatin structure prior to lineage commitment and before CD3 transcription. The quite limited enzymatic analysis of Flanagan is based o a region in the mouse that has no homology to any region as found in the human. Furthermore, the paper does not identify the regions as analyzed as suitable for the identification of DC3 lymphocytes.

Clevers et al. (in Clevers H, Lonberg N, Dunlap S, Lacy E, Terhorst C. An enhancer located in a CpG-island 3′ to the TCR/CD3-epsilon gene confers T lymphocyte-specificity to its promoter. EMBO J. 1989 September; 8(9):2527-35.) describe that the gene encoding the CD3-epsilon chain of the T cell receptor (TCR/CD3) complex is uniquely transcribed in all T lymphocyte lineage cells. The human CD3-epsilon gene, when introduced into the mouse germ line, was expressed in correct tissue-specific fashion. The gene was then screened for T lymphocyte-specific cis-acting elements in transient chloramphenicol transferase assays. The promoter (−228 to +100) functioned irrespective of cell type. A 1225 by enhancer with strict T cell-specificity was found in a DNase I hypersensitive site downstream of the last exon, 12 kb from the promoter. This site was present in T cells only. The CD3-epsilon enhancer did not display sequence similarity with the T cell-specific enhancer of CD3-delta, a related gene co-regulated with CD3-epsilon during intrathymic differentiation. The CD3-epsilon enhancer was unusual in that it constituted a CpG island, and was hypomethylated independent of tissue type. Two HTLV I-transformed T cell lines were identified in which the CD3-epsilon gene was not expressed, and in which the enhancer was inactive. In contrast to the preferred embodiment of the present invention, Clevers et al. analyze a remotely located enhancer region of 3′ to the TCR/CD3-epsilon gene.

Hamerman et al. (in: Hamerman J A, Page S T, Pullen A M. Distinct methylation states of the CD8 beta gene in peripheral T cells and intraepithelial lymphocytes. J Immunol. 1997 Aug 1;159(3):1240-6) distinguish between CD4 and CD8 T-lymphocytes.

EP 1 213 360 describes a method of identifying a cell, tissue or nucleus, comprising collecting information on the methylation pattern of DNA isolated from the cell, tissue or nucleus and analyzing the resultant information.

WO 2004/050706 describes a sub-group of T-cells, and relates to characteristics of regulatory T-cells which define them as such. The application also describes the uses of such T-cells, compositions comprising them, and chemokines which recruit them in the modulation of an immune response.

Finally, EP 1 826 279 describes a method, in particular an in vitro method, for identifying FoxP3-positive regulatory T cells, preferably CD25⁺ CD4⁺ regulatory T cells of a mammal, comprising analyzing the methylation status of at least one CpG position in the gene foxp3 or an orthologous or paralogous gene thereof, and the use of DNA-methylation analysis of the gene of the transcription factor FoxP3 for a detection and quality assurance and control of regulatory T cells.

While the measurement and determination of CD4 and CD8 cells is generally easy and is usually achieved through analyzing the expression of said antigens on the cellular surface, clinically, it remains challenging to determine these cell types, since for the commonly used FACS analysis the cell samples need to be freshly isolated or immediately fixated in order to keep the cell entities intact. Thus, the detection of T lymphocytes, while desirous, is problematic, particularly for routine applications.

In view of the above, it is an object of the present invention to provide an improved and in particular robust method based on DNA methylation analysis as a superior tool in order to more conveniently and reliably identify T-lymphocytes.

The present invention solves the above object by providing a method for identifying T-lymphocytes in a mammal, in particular in a sample derived from a mammal, comprising analysing the methylation status of at least one CpG position in one or more of the genes for CD3γ, -δ, and -ε, wherein a demethylation of at least one CpG position to at least 90% in said sample is indicative for a CD3⁺ T-lymphocyte cell, in particular a CD3⁺ CD4⁺, and/or CD3⁻ CD8⁺ T-lymphocyte cell.

The present invention is based on the surprising finding of the inventors that the identification of CD3 gene as a specific epigenetic marker can greatly facilitate the clinical routine application of the analysis of the above markers. In contrast to FACS and mRNA measurements, the respective measurement(s) can be done independent of purification, storage and to quite some extend also to tissue quality.

In another preferred embodiment of the method according to the present invention, said at least one CpG position in said sample is demethylated to more than 91% and preferably more than 92% and most preferred more than 95%.

This concept is based on specific demethylation of the CD3 regions in CD3 positive T-lymphocytes. Using a simple and precise quantitative PCR method, the inventors show that CD3 demethylation represents a surrogate marker for T-lymphocyte counts in blood or tissues. The present inventors have thus identified particular regions within the CD3 gene, which are functionally involved in, or mandatory associated with, the existence of CD3 positive T-lymphocytes. In one preferred embodiment one very good region is either the promoter or the TLSDR with e.g. the nucleotide sequence according to SEQ ID No. 1 and others, containing many CpG motifs, which display a differential methylation status when cells expressing CD3 in either CD4⁺ or CD8⁺ cells compared with all other cells, not expressing CD3 if, for example, the bisulphite sequencing method is used.

The inventors could demonstrate that in CD3⁺ cells the CpG motifs are almost completely demethylated (i.e. to more than 70%, preferably 80%, preferably, more than 90% and most preferred more than 95%), whereas the same motifs are completely methylated in all CD3⁻ cells. The differential methylation of the CpG motifs within the aforementioned region correlates with CD3 expression. Thus, determination of the methylation status of the CD3 locus could become a valuable tool to identify T-lymphocytes, such as will be required/or at least of some value for measuring T-lymphocytes in autoimmune diseases, transplant rejections, cancer, allergy, or just the T-lymphocytes related immune status in any envisionable context, when desired. The assays allows measurement if T-lymphocytes without purification or any staining procedures. It even reports in solid tumors or other solid tissues the number of cells demethylated in said region, thus showing the total amount of CD3 positive tumor infiltrating T-lymphocytes.

The inventors have shown that the potential for constitutive expression of CD3 in T-lymphocytes coincides with epigenetic, i.e., DNA methylation based regulation. DNA methylation is a biologically and chemically stable epigenetic modification, resulting in long-term gene expression changes. The inventors found demethylation at the human CD3 locus to be restricted to T-lymphocytes when tested against all major peripheral blood cell types and a selection of non-blood cells. These data indicated that epigenetic modifications in the CD3 locus serve as valuable marker for the identification of cells with the phenotype of T-lymphocyte, regardless of the expression of the specific delta or gamma sub-chains.

In another preferred aspect of the method according to the present invention the methylation status of at least one CpG position in the genes SLA2, CHRNA3, C16orf24, LCK, FASLG, CD7, SIT1, IL32, CXCR6, UBASH3A, GRAP2, ITGB7 and TXK is analysed in analogy to what is described herein for CD3. These genes thus also allow the unambiguous identification of all CD3 positive T lymphocytes. Thus, in a preferred embodiment of the method according to the present invention, said at least one CpG position is present in the 5′ region upstream from the transcription start, promoter region, intron, and/or exon/intron border within the gene(s) SLA2, CHRNA3, C16orf24, LCK, FASLG, CD7, SIT1, IL32, CXCR6, UBASH3A, GRAP2, ITGB7 and TXK.

In order to further unambiguously identify all CD8 cells, the present invention in another preferred aspect thereof provides the equivalent analysis of the genes GNGT2, CRTAM, IL2RB and ZBTB32 among the CD3 positive T lymphocytes, as these markers are capable to segregate between CD8 and CD4 positive cells. This analysis is preferably performed simultaneously or subsequently to the analysis for the CD3 phenotype of the T-lymphocytes.

Equivalently, FLJ00060, FLJ38379, PPP6C, CD226, ZBTB7B and TNFAIP8 are capable of positively identifying CD4 expressing cells in whole blood and segregate between CD4 and CD8 positive CD3 positive cells.

Another preferred aspect of the method according to the present invention is directed at the use of DNA-methylation analysis of the genes CD3γ/δ/ε, or SLA2, CHRNA3, C16orf24, LCK, FASLG, CD7, SIT1, IL32, CXCR6, UBASH3A, GRAP2, ITGB7 and TXK or GNGT2, CRTAM, IL2RB and ZBTB32 or FLJ00060, FLJ38379, PPP6C, CD226, ZBTB7B and TNFAIP8 for the detection and quality assurance and control of T lymphocytes.

In another preferred embodiment of the method according to the present invention, said at least one CpG position is present in the 5′ region upstream from the transcription start, promoter region, intron, and/or exon/intron border within the CD3 gene. The present invention also provides the surprising finding that in particularly preferred regions of the gene for CD3, the so-called “TLSDRs” (T lymphocyte specific demethylated regions), the CpG motifs are almost completely demethylated (i.e. to more than 90%, preferably 91%, preferably, more than 92% and most preferred more than 95%), whereas the same motifs are completely methylated in all non T lymphocytes. Thus, this regions and the diagnostic uses thereof also provides a valuable and reliable tool for a diagnostic analysis according to the present invention. The TLSDR according to the present invention are located in amplicon No. 1405 (SEQ ID No. 6), amplicon No. 1406 (SEQ ID No. 7), and/or amplicon No. 1408 (SEQ ID No. 8). All of these amplicons are parts of the overall region of interest of the present invention as depicted in SEQ ID No. 1.

In a preferred embodiment of the method according to the present invention, said analysis of the methylation status comprises amplification with at least one primer of suitable primer pairs that can be suitably designed based on SEQ ID No. 1, preferably oligomers according to any of SEQ ID No. 2 to 5.

Preferably, the amplification involves a polymerase enzyme, a PCR or chemical amplification reaction, or other amplification methods as known to the person of skill as described below, e.g. in the context of MSP, HeavyMethyl, Scorpion, MS-SNUPE, MethylLight, bisulfite sequencing, methyl specific restriction assays. With the amplification, the amplicon of the TLSDR or any other region in the CD3 gene or any paralog or ortholog as described herein is produced that is a particularly preferred “tool” for performing the method(s) according to the present invention. Consequently, an oligomer according to any of SEQ ID No. 2 to 5 or the amplicon as amplified by a primer pair as mentioned above constitute preferred embodiments of the present invention.

The person of skill will furthermore be able to select specific subsets of CpG positions in order to minimise the amount of sites to be analyzed, for example at least one of CpG position 1, 2, 3, 4, 5, 6, 7, 8, 9, and 10 of the amplicon No. 1405 (SEQ ID No. 6), amplicon No. 1406 (SEQ ID No. 7), and amplicon No. 1408 (SEQ ID No. 8), or all sites as present on the amplicons or according to SEQ ID No 1 or other sequences in the CD3 locus. The positions are numerically counted from the 5′-end of the amplicon as generated and analysed. Preferred are combinations of 4, 5, 6,- or 7 positions, which are producing enough information in order to be informative in the context of the present invention.

In order to analyze the methylation status of CpG positions, any known method to analyse DNA methylation can be used. In a preferred embodiment of the method according to the present invention, the analysis of the methylation status comprises a method selected from methylation specific enzymatic digests, bisulphite sequencing, analysis selected from promoter methylation, CpG island methylation, MSP, HeavyMethyl, MethyLight, Ms-SNuPE or other methods relying on a detection of amplified DNA. These methods are well known to the person of skill, and can be found in the respective literature.

In a preferred embodiment of the method according to the present invention, said method is suitable for routine application, for example on a DNA-chip. Based on the above information and the respective literature, the person of skill will be able to adjust the method as above to such settings.

In another preferred embodiment of the method according to the present invention, the identification comprises a distinction of said T-lymphocytes from all major peripheral blood cell types or non-blood cells, preferably, but not limited to, CD14, CD15, CD19, and CD56.

In yet another preferred embodiment of the method according to the present invention, the sample is selected from a mammalian body fluid, including human blood samples, or a tissue, organ or cell type blood sample, a sample of blood lymphocytes or a fraction thereof. Preferably, said mammal is a mouse, rat, monkey or human. The samples can be suitably pooled, if required.

Another preferred aspect of the method according to the present invention then further comprises the step of concluding on the immune status of said mammal based on said T-lymphocytes as identified. The general lymphocyte population can be quantified and either be used as a benchmark to relatively quantify further detailed subpopulations such as the CD3CD4CD25 positive regulatory T cells or it can be used to finally detect this population to determine the overall immune activity status.

In yet another preferred embodiment of the methods according to the present invention, the mammal suffers from or is likely to suffer from autoimmune diseases, transplant rejections, cancer, and/or allergy.

Another preferred aspect of the method according to the present invention is related to a method for monitoring the level of CD3⁺ CD4⁺, and/or CD3⁺ CD8⁺ T-lymphocytes in a mammal, comprising a method as above, and comparing the amount of T-lymphocytes as identified to an earlier sample taken from the same mammal, and/or to a control sample. In yet another preferred embodiment of the methods according to the present invention, the mammal suffers from or is likely to suffer from autoimmune diseases, transplant rejections, cancer, and/or allergy.

Another preferred aspect of the method according to the present invention then relates to a method as above, further comprising measuring and/or monitoring the amount of said the amount of T-lymphocytes in response to chemical and/or biological substances that are provided to said mammal.

Another preferred aspect of the method according to the present invention relates to an oligomer according to any of SEQ ID No. 2 to 5 or an amplicon as amplified by a primer pair based on SEQ ID No. 1 and designed as described above, in particular the amplicon No. 1405 (SEQ ID No. 6), amplicon No. 1406 (SEQ ID No. 7), and amplicon No. 1408 (SEQ ID No. 8).

Yet another preferred aspect of the present invention then relates to a kit for identifying and/or monitoring CD3⁺ CD4⁺, and/or CD3⁺ CD8⁺ T-lymphocytes in a mammal based on the analysis of the methylation status of CpG positions in the gene CD3, comprising materials for performing a method according the present invention as described herein. Preferably, said kit comprises a) a bisulfite reagent, and b) materials for the methylation analysis of CpG positions as comprised by the amplicon No. 1405 (SEQ ID No. 6), amplicon No. 1406 (SEQ ID No. 7), and amplicon No. 1408 (SEQ ID No. 8). Further preferred, the positions consist of positions 1, 2, 3, 4, 5, 6, 7, 8, 9, and 10 of said amplicons. Most preferred are 5, 6, or 7 or all positions on said amplicon.

Finally, the present invention also encompasses the use of an oligomer or amplicon or a kit according to the present invention for identifying and/or for monitoring CD3⁺ CD4⁺, and/or CD3⁺ CD8⁺ T-lymphocytes in a mammal.

In summary, using the CD3 marker, the inventors very specifically identified (but not differentiated) both CD3/CD4 positive as well as CD3/CD8 positive T lymphocytes. Using the marker CD8beta, for example CD8 positive lymphocytes could then be distinguished from CD4 lymphocytes. This, when using a combination of the present marker(s) and the CD8beta marker, CD4 and CD8 cells can be specifically distinguished. This was not possible before the invention, since all CD4 looked identical to non-T lymphocytes.

The invention will now be further described based on the following examples and with reference to the accompanying figures and the sequence protocol, without being limited thereto. For the purposes of the present invention, all references as cited herein are incorporated by reference in their entireties. In the Figures and Sequences,

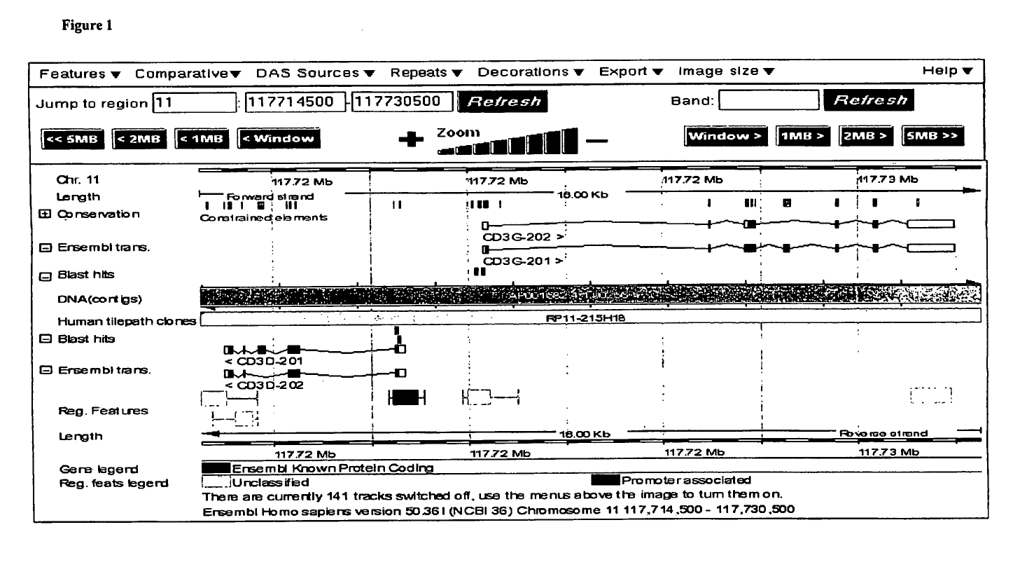

FIG. 1 shows the differentially methylated gene region as found for CD3 g and d and are indicated by red lines as “blast hits”.

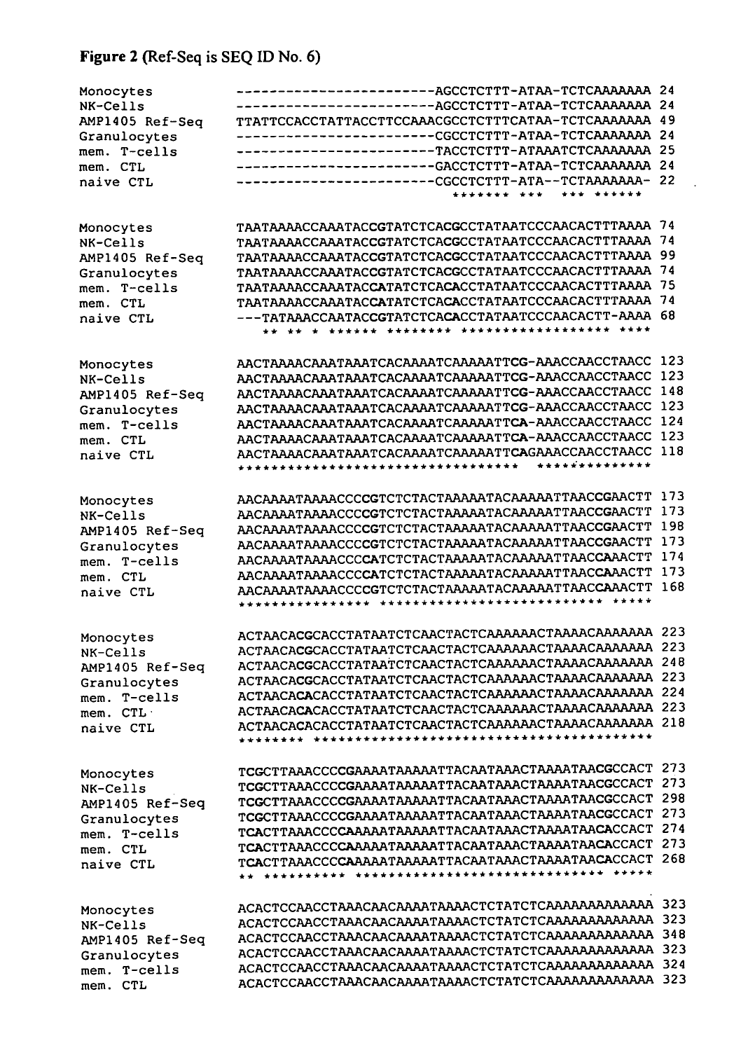

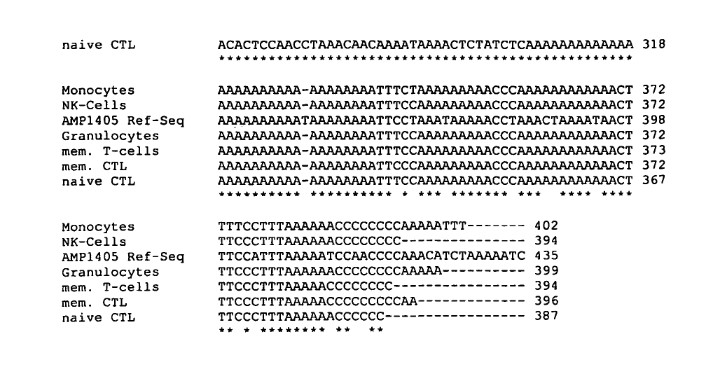

FIG. 2 shows the analysis of amplicon No. 1405 (SEQ ID No. 6), bisulfite strand 2, sequencing direction: forward. Relevant positions are indicated in bold.

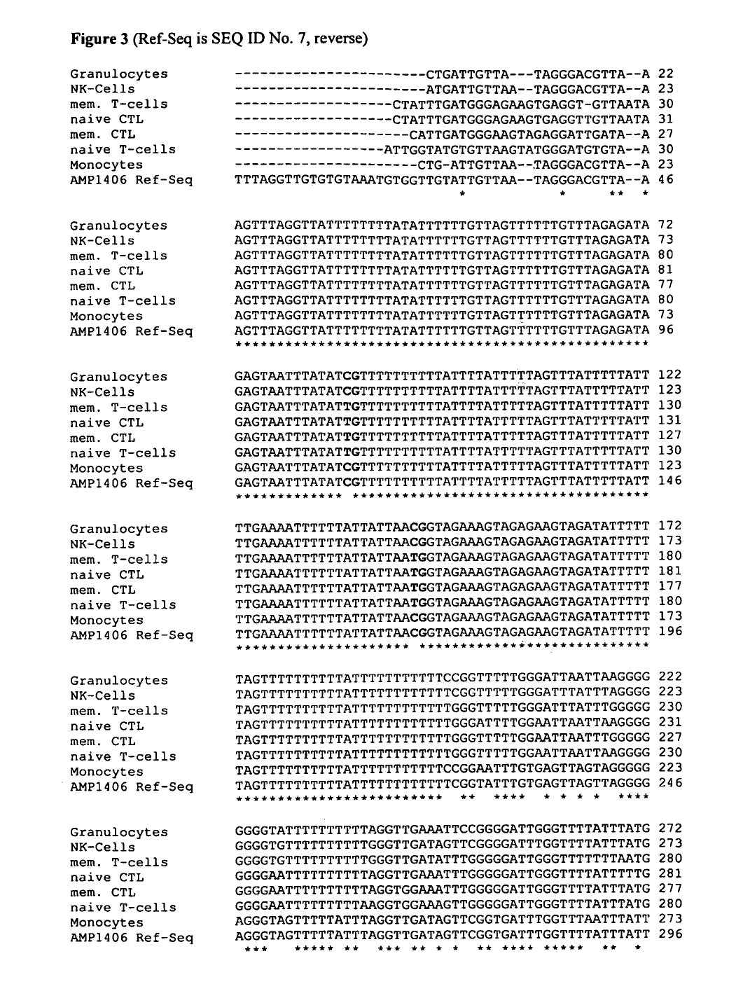

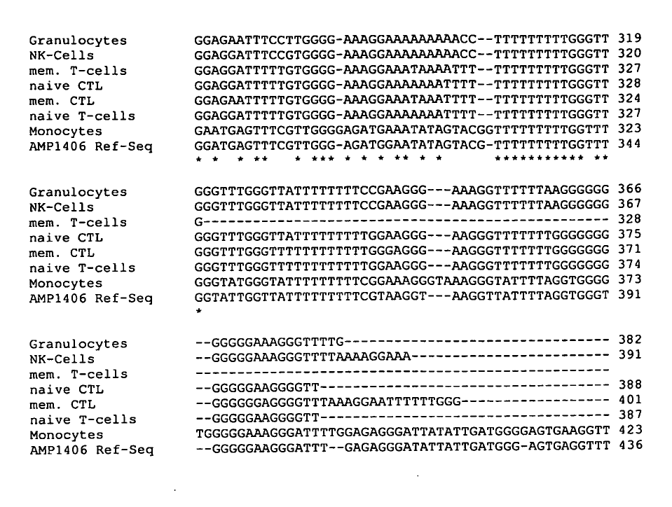

FIG. 3 shows the analysis of amplicon No. 1406, bisulfite strand 2, sequencing direction: reverse. Relevant positions are indicated in bold.

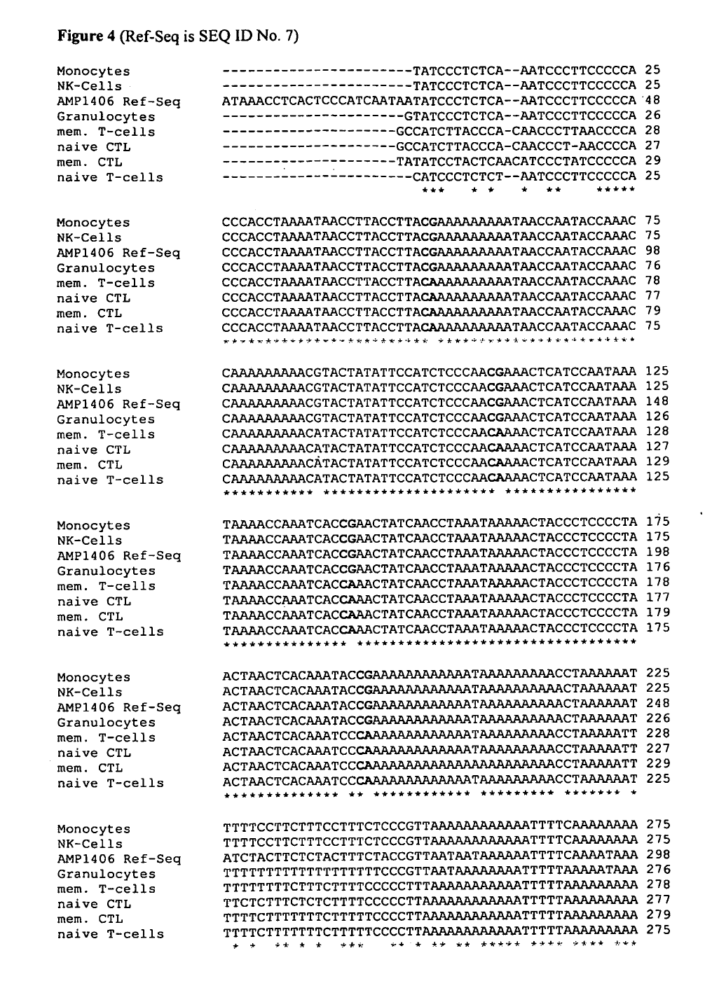

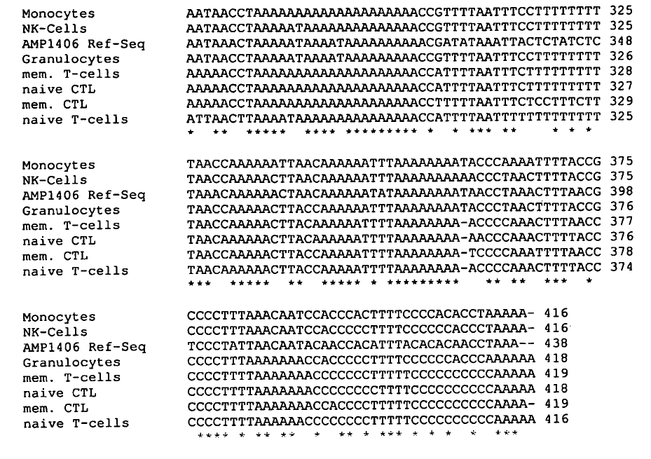

FIG. 4 shows the analysis of amplicon No. 1406 (SEQ ID No. 7), bisulfite strand: 2, sequencing direction: forward. Relevant positions are indicated in bold.

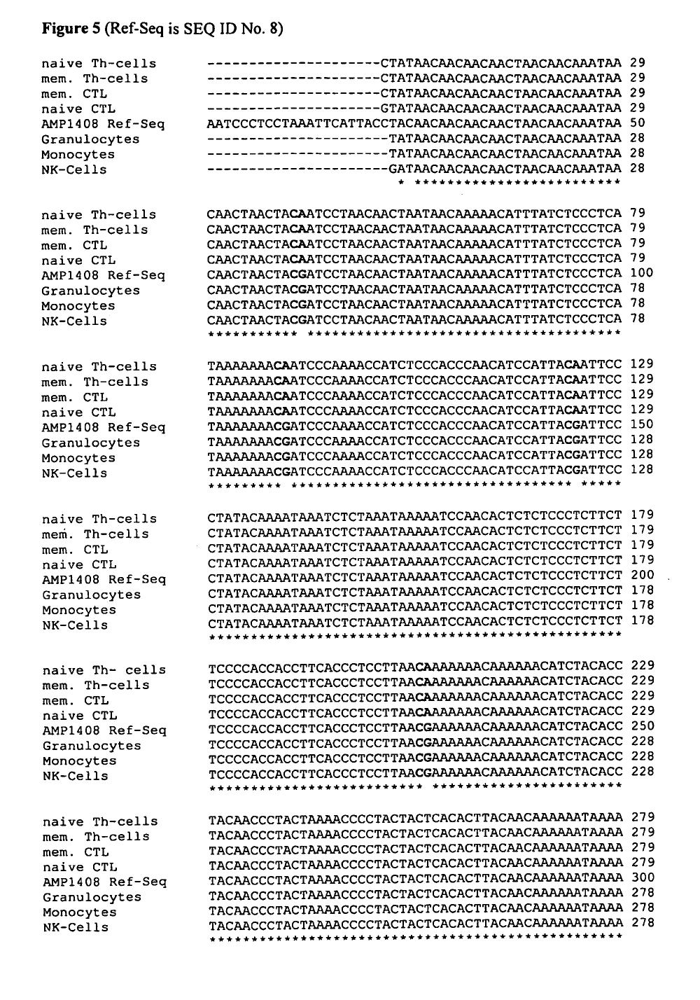

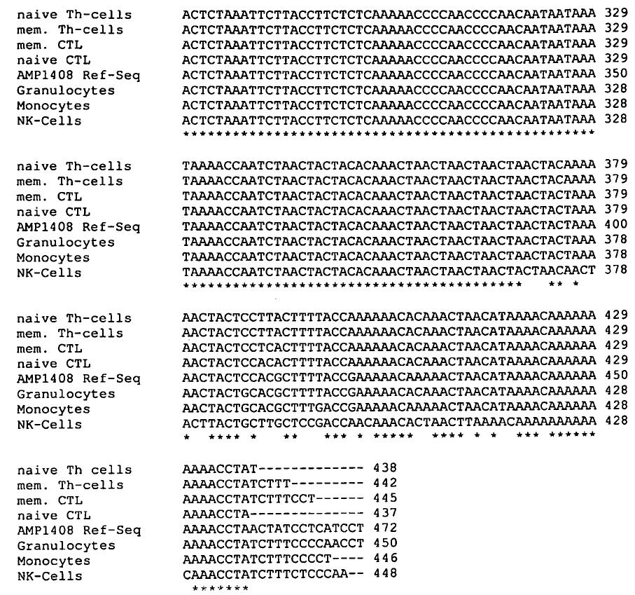

FIG. 5 shows the analysis of amplicon No. 1408 (SEQ ID No. 8), bisulfite strand: 2, sequencing direction: forward. Relevant positions are indicated in bold.

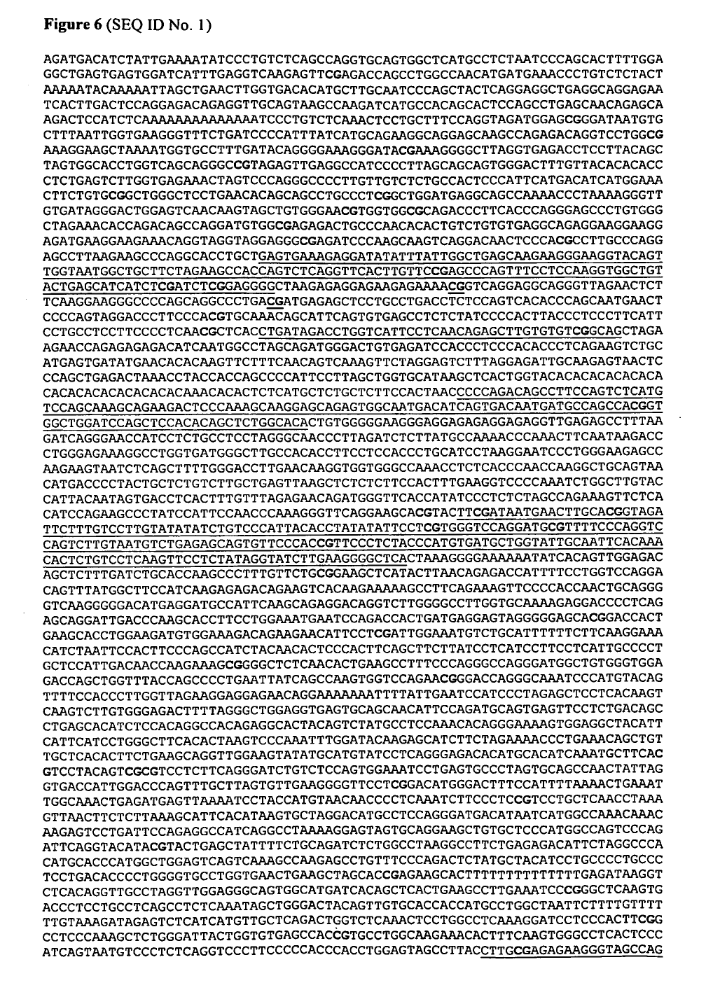

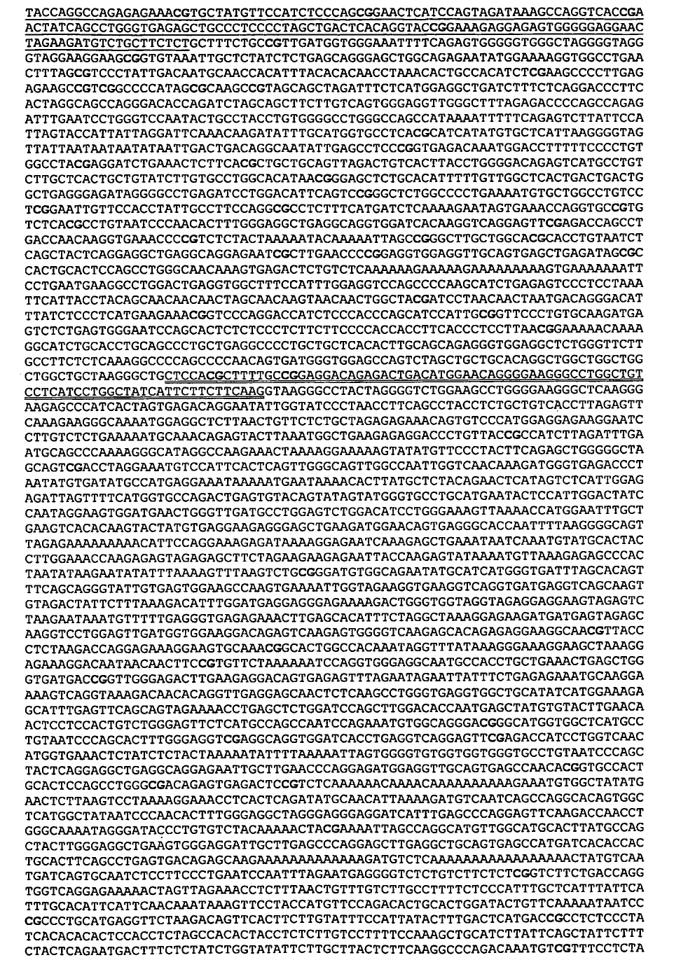

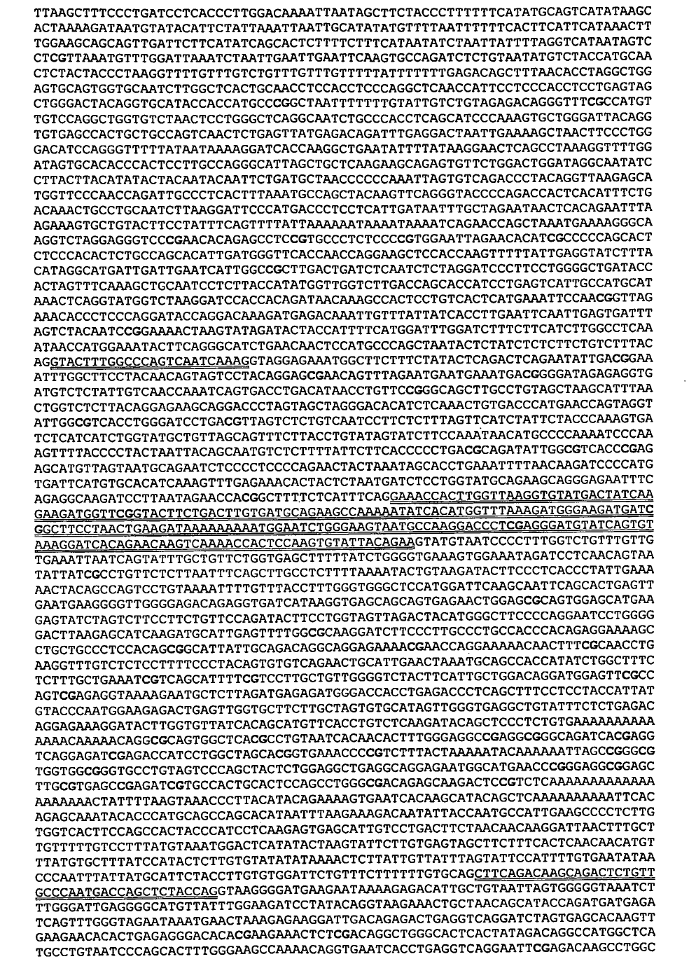

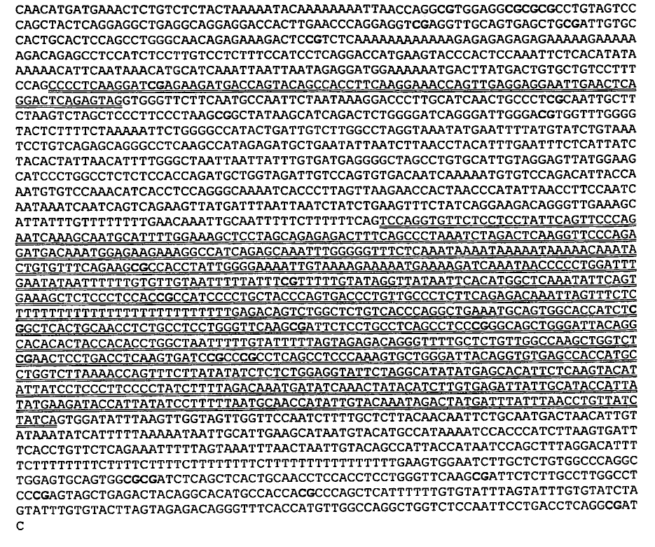

FIG. 6 shows the regulatory regions of the γ,δ -T-cell receptor, including regions Nr. 1405 1406 and 1408 on chromosome 11 (NCBI36:11:117714000:117730500:1). Exon sequences are underlined for CD3ε, and double underlined for CD3γ. CGs are in bold.

SEQ ID No. 1 shows the sequence of the region as considered to have a specific methylation pattern in CD3 cells.

SEQ ID No. 2 to 5 and 9 show the sequences of oligonucleotides used in the Illumina Chip-Fragment assay for CD3γ and CD3δ and CD3ε.

SEQ ID No. 6 to 8 show the reference sequences of amplicon No. 1405 (SEQ ID No. 6), amplicon No. 1406 (SEQ ID No. 7), and amplicon No. 1408 (SEQ ID No. 8).

SEQ ID No. 10 to 38 show the sequences around the CpG positions as analyzed in other preferred T-lymphocyte markers of the present invention according to example 2.

SEQ ID No. 39 to 65 show the sequences as depicted in the alignments of FIGS. 2 to 5. If not stated otherwise, the sequences are ordered in the succession of their appearance in FIGS. 2 to 5.

### EXAMPLES

### Example 1

### CD3-Analysis

The inventors have purified various blood subsets, including CD3/CD4, CD3/CD8 naïve and memory T lymphocytes, CD56 natural killer cells, CD19 naïve and memory B cells, CD14 monocytes and CD15 granulocytes. DNA from the purified cells was bisulfite-treated and analysed at various CpG dinucleotide motifs. The inventors then compared the methylation status (finding C as for Cytosine that was methylated in the original sequence versus T for cytosine that was unmethylated in the original sequence).

The data showed various CpG motifs and areas in the CD3γ, δ and ε that were demethylated in all CD3CD4 and CD3CD8 cell types while methylated in all other blood cell types. The differentially methylated gene regions as found for CD3γ, δ and ε are shown below in FIG. 1 and are indicated in bold as “blast hits”

The data, as observed with Illumina Golden Gate technology, show that all CD4 and CD8 positive memory (0.06 and 0.06 respectively) and naive (0.03 and 0.06, respectively) T cells are subject to much lower methylation rates than all other tested cell types, including CD15 (0.92), CD14 (0.90), CD19 memory and naïve (0.81 and 0.67, respectively), CD56 (0.86) positive blood cells as well as cells derived (0.78) from non-blood tissues. The experimental results are further depicted in the following table.

TABLE 1
Methylation analysis results

| | | S1 | S2 | S3 | S4 | S5 | S6 | S7 | S8 | S9 | S10 | S11 | S12 |
|---|---|---|---|---|---|---|---|---|---|---|---|---|---|
| | | n = 12; | n = 5; | Pool; | Pool; | Pool; | Pool; | Pool; | Pool; | Pool; | Pool; | Pool; | Pool; |
| Gene | Chr. | female | male | female | (5 male) | (5 male) | (5 male) | (5 male) | (5 male) | (5 male) | (5 male) | (5 male) | (5 male) |
| CD3D | 11 | 0.8 | 0.6 | 0.6 | 0.9 | 0.9 | 0.9 | 0.0 | 0.1 | 0.1 | 0.1 | 0.7 | 0.8 |
| | | 0.9 | 0.7 | 0.7 | 0.9 | 0.9 | 0.9 | 0.1 | 0.1 | 0.2 | 0.2 | 0.7 | 0.8 |
| CD3G | 11 | 0.7 | 0.6 | 0.7 | 0.9 | 0.9 | 0.8 | 0.1 | 0.1 | 0.1 | 0.1 | 0.8 | 0.9 |
| | | 0.4 | 0.6 | 0.5 | 0.7 | 0.8 | 0.7 | 0.1 | 0.1 | 0.1 | 0.1 | 0.3 | 0.6 |

S1 = ovarian tissue,
S2 = whole blood,
S3 peripheral blood mononuclear cells,
S4 = granulocytes,
S5 = monocytes,
S6 = NK cells,
S7 = naïve T helper cells,
S8 = memory T helper cells,
S9 = naïve cytotoxic T cells,
S10 = memory cytotoxic T cells,
S11 = naïve B-cells,
S12 = memory B-cells.,
Chr = chromosome

The data showing the high specificity were obtained using a IIlumina Golden Gate technology, with the following genomic CpGs regions analysed

CD3γ
cg15880738
(SEQ ID No. 2)
(+1)
AGCTGCTGCACAGGCTGGCTGGCTGGCTGGCTGCTAAGGGCTGCTCCACG
cg07545925
(SEQ ID No. 3)
(+1)
CGGAAAAACAAAAGGCATCTGCACCTGCAGCCCTGCTGAGGCCCCTGCTG
CD3δ
cg24841244
(SEQ ID No. 4)
(−1)
ACCCAGGCTGATAGTTCGGTGACCTGGCTTTATCTACTGGATGAGTTCCG
cg07728874
(SEQ ID No. 5)
(−1)
TGGAACATAGCACGTTTCTCTCTGGCCTGGTACTGGCTACCCTTCTCTCG
CD3ε
cg24612198
(Seq ID No. 9)
AGTCATCTGTTTTGCTTTTTTTCCAGAAGTAGTAAGTCTGCTGGCCTCCG

Since the Illumina technology does only allow the analysis of a single CpG (or rather 2 CpGs per gene locus) the inventors verified the methylation properties using bisulfite sequencing. For that, the inventors bisulfite treated the samples using the Qiagen EpiTect kit, and sequenced the samples using an ABI 3100 prism sequencer. For data interpretation, the KB base calling software supplied by ABI was used.

While not providing entirely quantitative results calculating the percentile of methylated (i.e., CG signal on the plus strand sequence) versus unmethylated (i.e., TG on the plus strand sequence), the data were unambiguous for the purified cell types. All T-lymphocytes were overwhelmingly demethylated at all CG positions analysed, whereas all other analysed cell types were methylated at identical positions.

### Example 2

### Analysis of Additional Markers in Analogy to CD3

In order to identify further suitable markers distingushing and monitoring T-lymphocytes, other markers in addition to CD3 have been identified and tested through methylation analysis. It was found that methylation in the CpG positions in the genes for SLA2, CHRNA3, C16orf24, LCK, FASLG, CD7, SIT1, IL32, CXCR6, UBASH3A, GRAP2, ITGB7 and TXK can also be used in the context of the present invention, as these markers are also able to identify CD3 positive T lymphocytes.

Furthermore, other markers have been identified that identify the subset of the CD8 and CD4 positive cells in the group of CD3 positive T lymphocytes. The genes for GNGT2, CRTAM, IL2RB and ZBTB32 have been found to segregate between CD8 and CD4 positive cells. Equivalently, F1100060, FLJ38379, PPP6C, CD226, ZBTB7B and TNFAIP8 are capable of positively identifying CD4 expressing cells in whole blood and segregate between CD4 and CD8 positive CD3 positive cells.

The following table 2 summarizes the Illumina-data as obtained for the above markers at selected CpG positions. It can be seen that several other markers can be used in order to selectively identify CD4+ (for CD3+ lymphocyte subset-identification), namely FLJ00060; FLJ38379; PPP6C; CD226; ZBTB7B and/or TNFAIP8, in order to selectively identify CD8+ (for CD3+ lymphocyte subset-identification), namely GNGT2; CRTAM; IL2RB and/or ZBTB32, and in order to selectively identify CD3+ lymphocytes, namely CD3D; CD3G, and/or CD3E, and/or SLA2,CHRNA3, C16orf24; LCK; FASLG; FASLG; CD7; SIT1; IL32; CXCR6; UBASH3A; GRAP2; ITGB7 and/or TXK, as shown with selected CpG sites as preferred examples. Based on the table, the person of skill will be able to extend the teaching regarding CD3 as herein to these markers and their CpG sites.

TABLE 2
Illumina-data as obtained selected markers at CpG positions Chr. = chromosome

| | | | SEQ ID | Ovar | | | BCST18 | BCST19 | |
|---|---|---|---|---|---|---|---|---|---|
| CD4+ | | | No./ | Tissue | Whole | PBMC | Granu- | Mono- | BCST20 |
| marker | Gene | | Accession | (mean, | Blood | (Pro- | locyte | cyte | NK |
| CpG-ID | Name | Chr. | No | n = 12) | (Pool) | mega) | (CD15+) | (CD14+) | (CD56+) |
| cg03602500 | FLJ00060 | 19 | 10/ | 0.802 | 0.728 | 0.779 | 0.858 | 0.851 | 0.873 |
| | | | NM_033206.1 | | | | | | |
| cg16173109 | FLJ38379 | 2 | 11/ | 0.614 | 0.675 | 0.719 | 0.833 | 0.844 | 0.784 |
| | | | XR_001026.1 | | | | | | |
| cg00620024 | PPP6C | 9 | 12/ | 0.641 | 0.619 | 0.692 | 0.801 | 0.851 | 0.836 |
| | | | NM_002721.3 | | | | | | |
| cg13164537 | CD226 | 18 | 13/ | 0.458 | 0.470 | 0.508 | 0.582 | 0.557 | 0.601 |
| | | | NM_006566.1 | | | | | | |
| cg01782486 | ZBTB7B | 1 | 14/ | 0.789 | 0.725 | 0.720 | 0.823 | 0.809 | 0.886 |
| | | | NM_015872.1 | | | | | | |
| cg07086380 | TNFAIP8 | 5 | 15/ | 0.728 | 0.579 | 0.635 | 0.857 | 0.733 | 0.793 |
| | | | NM_014350.1 | | | | | | |
| | | | SEQ ID | Ovar | | | BCST18 | BCST19 | |
| CD8+ | | | No./ | Tissue | Whole | PBMC | Granu- | Mono- | BCST20 |
| marker | Gene | | Accession | (mean, | Blood | (Pro- | locyte | cyte | NK |
| CpG-ID | Name | Chr | No | n = 12) | (Pool) | mega) | (CD15+) | (CD14+) | (CD56+) |
| cg17839611 | GNGT2 | 17 | 16/ | 0.779 | 0.629 | 0.688 | 0.828 | 0.867 | 0.207 |
| | | | NM_031498.1 | | | | | | |
| cg22512531 | CRTAM | 11 | 17/ | 0.651 | 0.634 | 0.709 | 0.835 | 0.735 | 0.5 |
| | | | NM_019604.2 | | | | | | |
| cg26757673 | IL2RB | 22 | 18/ | 0.799 | 0.662 | 0.752 | 0.878 | 0.9 | 0.092 |
| | | | NM_000878.2 | | | | | | |
| cg08539991 | ZBTB32 | 19 | 19/ | 0.823 | 0.736 | 0.791 | 0.912 | 0.926 | 0.674 |
| | | | NM_014383.1 | | | | | | |
| CD4+/ | | | SEQ ID | Ovar | | | BCST18 | BCST19 | |
| CD8+ | | | No./ | Tissue | Whole | PBMC | Granu- | Mono- | BCST20 |
| marker | Gene | | Accession | (mean, | Blood | (Pro- | locyte | cyte | NK |
| CpG-ID | Name | Chr | No | n = 12) | (Pool) | mega) | (CD15+) | (CD14+) | (CD56+) |
| cg24841244 | CD3D | 11 | 20/ | 0.783 | 0.582 | 0.644 | 0.917 | 0.904 | 0.857 |
| | | | NM_000732.3 | | | | | | |
| cg07728874 | CD3D | 11 | 21/ | 0.854 | 0.683 | 0.727 | 0.911 | 0.901 | 0.881 |
| | | | NM_000732.3 | | | | | | |
| cg15880738 | CD3G | 11 | 22/ | 0.714 | 0.616 | 0.682 | 0.874 | 0.876 | 0.836 |
| | | | NM_000073.1 | | | | | | |
| cg07545925 | CD3G | 11 | 23/ | 0.370 | 0.552 | 0.545 | 0.737 | 0.771 | 0.739 |
| | | | NM_000073.1 | | | | | | |
| cg24612198 | CD3E | 11 | 24/ | 0.679 | 0.485 | 0.563 | 0.794 | 0.793 | 0.698 |
| | | | NM_000733.2 | | | | | | |
| cg04759756 | SLA2 | 20 | 25/ | 0.857 | 0.673 | 0.754 | 0.892 | 0.925 | 0.711 |
| | | | NM_032214.2 | | | | | | |
| cg22670733 | CHRNA3 | 15 | 26/ | 0.792 | 0.733 | 0.721 | 0.911 | 0.888 | 0.8 |
| | | | NM_000743.2 | | | | | | |
| cg09830866 | C16orf24 | 16 | 27/ | 0.493 | 0.629 | 0.647 | 0.842 | 0.79 | 0.076 |
| | | | NM_023933.1 | | | | | | |
| cg17078393 | LCK | 1 | 28/ | 0.807 | 0.575 | 0.666 | 0.920 | 0.884 | 0.26 |
| | | | NM_005356.2 | | | | | | |
| cg10161121 | FASLG | 1 | 29/ | 0.704 | 0.587 | 0.697 | 0.905 | 0.892 | 0.068 |
| | | | NM_000639.1 | | | | | | |
| cg00071250 | FASLG | 1 | 30/ | 0.659 | 0.498 | 0.602 | 0.871 | 0.838 | 0.075 |
| | | | NM_000639.1 | | | | | | |
| cg02473123 | CD7 | 17 | 31/ | 0.811 | 0.681 | 0.767 | 0.942 | 0.898 | 0.339 |
| | | | NM_006137.6 | | | | | | |
| cg15518883 | SIT1 | 9 | 32/ | 0.817 | 0.601 | 0.696 | 0.911 | 0.885 | 0.9033 |
| | | | NM_014450.2 | | | | | | |
| cg18350391 | IL32 | 16 | 33/ | 0.332 | 0.724 | 0.794 | 0.937 | 0.901 | 0.658 |
| | | | NM_001012631.1 | | | | | | |
| cg25226014 | CXCR6 | 3 | 34/ | 0.702 | 0.481 | 0.548 | 0.789 | 0.89 | 0.35 |
| | | | NM_006564.1 | | | | | | |
| cg13578652 | UBASH3A | 21 | 35/ | 0.564 | 0.444 | 0.475 | 0.537 | 0.703 | 0.169 |
| | | | NM_018961.2 | | | | | | |
| cg25712380 | GRAP2 | 22 | 36/ | 0.633 | 0.602 | 0.639 | 0.824 | 0.83 | 0.506 |
| | | | NM_004810.2 | | | | | | |
| cg19812619 | ITGB7 | 12 | 37/ | 0.819 | 0.651 | 0.785 | 0.874 | 0.897 | 0.645 |
| | | | NM_000889.1 | | | | | | |
| cg02600394 | TXK | 4 | 38/ | 0.775 | 0.576 | 0.682 | 0.867 | 0.909 | 0.082 |
| | | | NM_003328.1 | | | | | | |
| | | | BCST23 | BCST24 | | | | | |
| | BCST21 | BCST22 | CTL | CTL | | | Mean | | |
| | T naive | T mem | naive | mem | | | Value | | Delta |
| CD4+ | (CD4+ | (CD4+ | (CD8+ | (CD8+ | BCST25 | BCST26 | Target | Mean | Meth |
| marker | CD27+ | CD27+ | CD27+ | CD27+ | B naive | B mem | Cell | Value | (Target- |
| CpG-ID | CD45RA+) | CD45RA−) | CD45RA+) | CD45RA−) | (CD19+) | (CD19+) | Type | Rest | Rest) |
| cg03602500 | 0.235 | 0.279 | 0.521 | 0.687 | 0.656 | 0.747 | 0.257 | 0.75 | −0.493 |
| cg16173109 | 0.1141 | 0.113 | 0.341 | 0.383 | 0.418 | 0.385 | 0.113 | 0.599 | −0.486 |
| cg00620024 | 0.107 | 0.169 | 0.269 | 0.420 | 0.404 | 0.502 | 0.138 | 0.603 | −0.465 |
| cg13164537 | 0.029 | 0.071 | 0.351 | 0.543 | 0.366 | 0.501 | 0.05 | 0.494 | −0.443 |
| cg01782486 | 0.190 | 0.428 | 0.875 | 0.864 | 0.586 | 0.414 | 0.309 | 0.749 | −0.44 |
| cg07086380 | 0.110 | 0.106 | 0.288 | 0.148 | 0.309 | 0.208 | 0.108 | 0.528 | −0.419 |
| | | | BCST23 | BCST24 | | | | | |
| | BCST21 | BCST22 | CTL | CTL | | | Mean | | |
| | T naive | T mem | naive | mem | | | Value | | Delta |
| CD8+ | (CD4+ | (CD4+ | (CD8+ | (CD8+ | BCST25 | BCST26 | Target | Mean | Meth |
| marker | CD27+ | CD27+ | CD27+ | CD27+ | B naive | B mem | Cell | Value | (Target- |
| CpG-ID | CD45RA+) | CD45RA−) | CD45RA+) | CD45RA−) | (CD19+) | (CD19+) | Type | Rest | Rest) |
| cg17839611 | 0.340 | 0.562 | 0.09 | 0.119 | 0.854 | 0.722 | 0.104 | 0.648 | −0.543 |
| cg22512531 | 0.363 | 0.630 | 0.079 | 0.082 | 0.194 | 0.336 | 0.08 | 0.559 | −0.478 |
| cg26757673 | 0.573 | 0.086 | 0.157 | 0.061 | 0.411 | 0.403 | 0.109 | 0.555 | −0.446 |
| cg08539991 | 0.563 | 0.153 | 0.303 | 0.085 | 0.648 | 0.140 | 0.194 | 0.637 | −0.442 |
| | | | BCST23 | BCST24 | | | | | |
| | BCST21 | BCST22 | CTL | CTL | | | Mean | | |
| CD4+/ | T naive | T mem | naive | mem | | | Value | | Delta |
| CD8+ | (CD4+ | (CD4+ | (CD8+ | (CD8+ | BCST25 | BCST26 | Target | Mean | Meth |
| marker | CD27+ | CD27+ | CD27+ | CD27+ | B naive | B mem | Cell | Value | (Target- |
| CpG-ID | CD45RA+) | CD45RA−) | CD45RA+) | CD45RA−) | (CD19+) | (CD19+) | Type | Rest | Rest) |
| cg24841244 | 0.025 | 0.057 | 0.057 | 0.058 | 0.673 | 0.810 | 0.049 | 0.771 | −0.721 |
| cg07728874 | 0.128 | 0.149 | 0.175 | 0.155 | 0.744 | 0.807 | 0.152 | 0.814 | -0.661 |
| cg15880738 | 0.061 | 0.091 | 0.093 | 0.103 | 0.811 | 0.898 | 0.087 | 0.788 | −0.701 |
| cg07545925 | 0.133 | 0.149 | 0.132 | 0.131 | 0.303 | 0.601 | 0.132 | 0.49 | −0.358 |
| cg24612198 | 0.064 | 0.036 | 0.093 | 0.051 | 0.206 | 0.279 | 0.061 | 0.562 | −0.501 |
| cg04759756 | 0.363 | 0.228 | 0.129 | 0.211 | 0.756 | 0.849 | 0.233 | 0.802 | −0.569 |
| cg22670733 | 0.122 | 0.180 | 0.092 | 0.315 | 0.456 | 0.668 | 0.177 | 0.746 | −0.568 |
| cg09830866 | 0.041 | 0.056 | 0.033 | 0.058 | 0.72 | 0.510 | 0.047 | 0.588 | −0.541 |
| cg17078393 | 0.05 | 0.037 | 0.036 | 0.041 | 0.144 | 0.27 | 0.041 | 0.566 | −0.524 |
| cg10161121 | 0.051 | 0.06 | 0.088 | 0.06 | 0.353 | 0.441 | 0.065 | 0.581 | −0.516 |
| cg00071250 | 0.061 | 0.069 | 0.139 | 0.051 | 0.391 | 0.4144 | 0.08 | 0.543 | −0.463 |
| | | | | | | 6208 | | | |
| cg02473123 | 0.102 | 0.328 | 0.168 | 0.372 | 0.767 | 0.81 | 0.243 | 0.752 | −0.509 |
| cg15518883 | 0.11 | 0.142 | 0.154 | 0.358 | 0.392 | 0.373 | 0.191 | 0.697 | −0.506 |
| cg18350391 | 0.296 | 0.169 | 0.15 | 0.192 | 0.881 | 0.406 | 0.202 | 0.704 | −0.502 |
| cg25226014 | 0.028 | 0.046 | 0.046 | 0.193 | 0.258 | 0.514 | 0.078 | 0.567 | −0.488 |
| cg13578652 | 0.027 | 0.04 | 0.035 | 0.05 | 0.602 | 0.673 | 0.038 | 0.521 | −0.483 |
| cg25712380 | 0.097 | 0.093 | 0.089 | 0.1 | 0.179 | 0.310 | 0.095 | 0.565 | −0.470 |
| cg19812619 | 0.3374 | 0.275 | 0.199 | 0.177 | 0.608 | 0.4337 | 0.247 | 0.714 | −0.467 |
| cg02600394 | 0.036 | 0.075 | 0.051 | 0.354 | 0.376 | 0.484 | 0.129 | 0.594 | −0.465 |

1. A method for identifying T-lymphocytes in a sample derived from a mammal, comprising analysing the methylation status of at least one CpG position in one or more of the genes for CD3γ, -δ, and -ε, or SLA2, CHRNA3, C16orf24, LCK, FASLG, CD7, SIT1, IL32, CXCR6, UBASH3A, GRAP2, ITGB7 or TXK, wherein a demethylation of at least one CpG position to at least 90% in said sample is indicative for a CD3⁺ T-lymphocyte cell.

2. The method according to claim 1, wherein said at least one CpG position in said sample is demethylated to more than 91%.

3. The method according to claim 1, wherein said at least one CpG position is present in the 5′ region upstream from the transcription start, promoter region, intron, and/or exon/intron border within the CD3 genetic region.

4. The method according to claim 1, wherein said at least one CpG position is found in amplicon No. 1405 according to SEQ ID NO: 6, amplicon No. 1406 according to SEQ ID NO: 7, and/or amplicon No. 1408 according to SEQ ID NO: 8.

5. The method according to claim 1, wherein the analysis of the methylation status comprises a method selected from methylation specific enzymatic digests, bisulphite sequencing, analysis selected from promoter methylation, CpG island methylation, MSP, HeavyMethyl, MethyLight, Ms-SNuPE or other methods relying on a detection of amplified DNA.

6. The method according to claim 1, further comprising a methylation analysis of at least one CpG position in the genes for CD4⁺ and/or CD8 or in the genes for GNGT2, CRTAM, IL2RB, or ZBTB32, or FLJ00060, FLJ38379, PPP6C, CD226, ZBTB7B, or TNFAIP8.

7. The method according to claim 1, wherein said method is suitable for application on a DNA-chip.

8. The method according to claim 1 wherein said identification comprises a distinction of said T-lymphocytes from all major peripheral blood cell types or non-blood cells.

9. The method according to claim 1, wherein said sample is selected from a mammalian blood sample, or a tissue, organ or cell type blood sample, a sample of blood lymphocytes or a fraction thereof

10. The method according to claim 1, wherein said mammal is a mouse, rat, monkey or human.

11. The method according to claim 1, further comprising the step of determining the immune status of said mammal based on said T-lymphocytes as identified.

12. The method according to claim 1 wherein said mammal suffers from or is likely to suffer from an autoimmune disease, a transplant rejection, cancer, and/or an allergy.

13. A method for monitoring the level of CD3⁺ T-lymphoeytes in a mammal, comprising a method according to claim 1, and further comparing the amount of T-lymphocytes as identified to an earlier sample taken from the same mammal, and/or to a control sample.

14. The method according to claim 13, wherein said mammal suffers from or is likely to suffer from an autoimmune disease, a transplant rejection, cancer, and/or an allergy.

15. The method according to claim 12, further comprising measuring and/or monitoring the amount of said T-lymphocytes in response to a chemical and/or biological substance that is provided to said mammal.

16. An oligomer of SEQ ID NO: 2 to 5 or the amplicon Nr. 1404 according to FIG. 2, 1406 according to FIG. 3, or 1408 according to FIG. 5.

17. A kit for identifying and/or monitoring CD3⁺ T-lymphocytes in a mammal based on the analysis of the methylation status of CpG positions in the gene CD3, comprising materials for performing a method according to claim 1.

18. The kit according to claim 16, comprising a) a bisulfite reagent, and b) materials for the methylation analysis of CpG positions selected from the positions consisting of positions 1, 2, 3. 4, 5, 6, 7, 8, 9, and 10 of the amplicon Nr. 1405 according to SEQ ID NO: 6, amplicon No. 1406 according to SEQ ID NO: 7, and amplicon No. 1408 according to SEQ ID NO: 8.

19. A method for identifying and/or monitoring CD3⁺ CD4⁺, or CD3⁺ CD8⁺ T-lymphocytes in a mammal, wherein said method comprises the use of an oligomer or amplicon according to claim 16.
